(19) United States
(12) Patent Application Publication
(10) Pub. No.: US 2022/0090096 A1
(43) Pub. Date: Mar. 24, 2022

(54) GENETIC CONSTRUCT

(71) Applicant: THE UNIVERSITY OF NOTTINGHAM, Notingham (GB)

(72) Inventors: Nigel Minton, Nottingham (GB); Ines Canadas Blasco, Nottingham (GB); Daphne Groothuis, Nottingham (GB); Maria Zygouropoulou, Nottingham (GB)

(21) Appl. No.: 17/426,095

(22) PCT Filed: Jan. 28, 2020
(86) PCT No.: PCT/GB2020/050192

§ 371 Date: Jul. 27, 2021

(30) Foreign Application Priority Data
Jan. 28, 2019 (GB) 1901165.9

(51) Int. Cl. C12N 15/74; C12N 9/22; C07K 14/33

(52) U.S. Cl. CPC C12N 15/74; C12N 9/22; C12N 2310/20; C12N 2800/101; C07K 14/33

(57) ABSTRACT

A genetic construct comprising a DNA polynucleotide sequence which encodes a riboswitch operably linked to a coding region, wherein the coding region encodes a target gene and the riboswitch modulates translation or transcription of the coding region. A vector or a host cell comprising the genetic construct of the invention. A method of controlling expression of a target gene in a cell using the genetic construct of the invention.

The present invention relates to a gene expression control system. In particular, to the use of a riboswitch to control the expression of a target gene, wherein when the riboswitch is not activated expression of the target gene is absent or very low, and when the riboswitch is activated the gene is expressed. Preferably the dynamic range of expression is low.

In some circumstances unwanted gene expression can be harmful or even toxic to a cell. Furthermore the regulation of expression of some genes is inherently leaky, that is the background level of gene expression, even without gene expression activation, can be relatively high or at least sufficient to cause harm to the cell. The aim of the present invention is to provide a gene expression control system which reduces or even eliminates background levels of gene expression and offers a tight regulation of gene expression.

According to a first aspect the invention provides a genetic construct comprising a DNA polynucleotide sequence which encodes a riboswitch operably linked to a coding region, wherein the coding region encodes a target gene and the riboswitch modulates translation or transcription of the coding region.

The target gene may be any gene of interest, but it is preferably a gene where background levels of expression, even at a low level, can cause harm to the cell.

The riboswitch in the genetic construct may reduce the background level of target gene expression by about 5%, 10%, 20%, 15%, 20%, 25%, 30%, 35%, 40%, 45%, 50%, 55%, 60%, 65%, 70%, 75%, 80%, 85%, 90%, 95% or more. It may eliminate detectable background expression.

In this context the term “background expression” refers to the level of protein produced by a target gene in a cell under normal circumstances when expression of the gene is not desired, or expression of the target gene is not activated with an inducer. This level of expression is also sometimes referred to as the “leaky” level of expression, occurring because the gene promoter allows some expression even when not specifically activated.

In the genetic construct of the invention preferably the dynamic range of expression between the riboswitch being off and the riboswitch being on, and gene expression being activated, is low. The dynamic range may be between about 10 and about 100 fold of the off level, assuming that the off level is detectable. Preferably the dynamic range is between about 10 and about 20 fold that of the off level.

The riboswitch is preferably 5′ to the coding region. The coding region typically comprises at its 5′ terminus an ATG start codon.

The riboswitch may be an RNA molecule, such as mRNA. The riboswitch may comprise or consist of an aptamer domain, which is capable of specifically binding to an inducer, and an expression platform, which undergoes a conformational change (in response to the binding of the inducer to the aptamer domain) that promotes translation of the coding region.

The riboswitch may modulate translation of a coding region to which it is operably linked in response to contact of the aptamer domain with an inducer. The riboswitch may modulate translation of the coding region, in response to contact with an inducer, by positively regulating translation of the coding region (i.e. promoting translation of the coding region) or negatively regulating translation of the coding region (i.e. inhibiting translation of coding region). In a preferred embodiment the inducer activates the riboswitch such that it promotes translation of the coding region.

The expression platform of the riboswitch may comprise a nucleotide sequence encoding a regulatory domain that can be used to modulate translation or transcription of the coding region. The regulatory domain may be a ribosome binding site (RBS), which is also referred to as the Shine-Dalgarno (SD) sequence. The SD sequence is complementary to the 3′ end of the 16S rRNA. In Clostridia and Bacillus the sequence of the 3′ end of the 16S rRNA sequence may be:
3′-AUCUUUCCUCCACUAGGUCGGCGUCCAAGAGGAUG-5,

and the consensus SD sequence may be:
5′-AAAGGAGGUGU-3′

which is followed by an initiation codon, most commonly AUG. In around 8% of cases the start site is GUG, whereas UUG and AUU are rare initiators present in autogenously regulated genes. The optimal spacing between the SD sequence and the start codon is 8 nt, but translation initiation is only severely affected if this distance is increased above 14 nt or reduced below 4 nt [Shine, J. and Dalgarno, L. (1975) Eur. J. Biochem. 57, 221.]. The skilled person would appreciate that in the absence of a functional RBS, ribosomes are incapable of binding to mRNA, and thus incapable of being translated into a protein.

In embodiments in which the riboswitch positively regulates translation of the coding region, the regulatory domain may, in the absence of an inducer, be sequestered by the expression platform, thus preventing binding of one or more ribosomes to the regulatory domain. Binding of the inducer to the aptamer domain may cause the expression platform to undergo a conformational change that releases (the formerly sequestered) regulatory domain, such that one or more ribosomes can bind to the regulatory domain and thus translate the coding region into a protein.

The riboswitch may alternatively act by blocking transcription of a coding region by creating a terminator, which in the presence of an inducer is removed.

The riboswitch may be activated by a non-natural or a natural agent which acts as the inducer.

The riboswitch may be a naturally-occurring riboswitch or a synthetic riboswitch. A naturally occurring riboswitch may be a riboswitch responsive to adenosylcobalamin, aquacobalamin, thiamin pyrophosphate, flavin mononucleotide, s-adenosylmethionine, molybdenum cofactor, tungsten cofactor, tetrahydrofolate, s-adenosylhomocysteine, guanine, adenine, prequeuosine-1-, 2′-deoxyguanosine, cyclic di-gmp, cyclic di-amp, cyclic amp-gmp, ztp, mg²⁺, mn²⁺, f⁻, ni²⁺/co²⁺, lysine, glycine, glutamine, glucosamine-6-phosphate, azaaromatics or guanidine.

A synthetic riboswitches may be a riboswitch responsive to tetracycline; neomycin; 2,4,6-trinitrotoluene (TNT); ammeline; 5-azacytosine; theophylline; pyrimido[4,5-d]pyrimidine-2,4,-diamine (PPDA); 2-aminopyrimido[4,5-d]pyrimidin-4(3H)-one-(PPAO) or 2,6-diamino preQ0-(DPQ0).

The aptamer domain of the riboswitch may specifically bind the inducer, such as, theophylline, and thus be referred to as a theophylline-responsive riboswitch. Theophylline is a purine that has high affinity for the aptamer domain of the theophylline-responsive riboswitch. The discriminatory capacity of the aptamer with respect to related purines, which are structurally similar, is very high. For example, the aptamer of the theophylline-responsive riboswitch has a binding affinity that is 10,000-fold greater for theophylline than that of caffeine, which only differs from theophylline with respect to a methyl group located at nitrogen atom N-7. The aptamer domain specific for theophylline can be used in to create a positive or a negative regulatory riboswitch.

The riboswitch may be activated by an inducer. The inducer may induce gene expression by binding to the aptamer of a riboswitch. Thus, in one embodiment, the inducer may be theophylline. However, the skilled person would appreciate that the inducer may be a molecule that is capable of specifically binding to an aptamer domain, such as adenosylcobalamin; aquacobalamin; thiamin pyrophosphate; flavin mononucleotide; s-adenosylmethionine; molybdenum cofactor; tungsten cofactor; tetrahydrofolate s-adenosylhomocysteine; guanine; adenine; prequeuosine-1-, 2′-deoxyguanosine; cyclic di-gmp; cyclic di-amp; cyclic amp-gmp; ztp; mg2+; mn2+; f−; ni2+/co2+; lysine; glycine; glutamine, glucosamine-6-phosphate, azaaromatics, guanidine; tetracycline; neomycin; 2,4,6-trinitrotoluene (TNT); ammeline; 5-azacytosine; theophylline; pyrimido[4,5-d]pyrimidine-2,4,-diamine (PPDA); 2-aminopyrimido[4,5-d]pyrimidin-4(3H)-one-(PPAO) or 2,6-diamino preQ0-(DPQ0). Similarly, the skilled person would appreciate that the aptamer domain may be any domain that specifically binds to an inducer.

In an embodiment, the riboswitch may be a positive regulatory theophylline-responsive riboswitch (i.e. a riboswitch that promotes translation of the coding region). The nucleotide sequence encoding the positive regulatory theophylline-responsive riboswitch may be as referred to herein as SEQ ID NO. 1, 2, 3, 4, 5, 6 or 7, as shown in Table 1.1. Riboswitches of SEQ ID NO. 1, 2 and 3 are known, whereas riboswitches of SEQ ID NO. 4, 5, 6 and 7 are new.

TABLE 1.1

| Ribo- | | Seq ID |
|---|---|---|
| switch | Sequence (5′→3′) | No: |
| D | GGUGAUACCAGCAUCGUCUUGAUGCCCUUGGCAGCACCC | 1 |
| | UGCUAAGGUAACAACAAGAUG | |
| E | GGUGAUACCAGCAUCGUCUUGAUGCCCUUGGCAGCACCC | 2 |
| | UGCUAAGGAGGUAACAACAAGAUG | |
| E* | GGUGAUACCAGCAUCGUCUUGAUGCCCUUGGCAGCACCC | 3 |
| | UGCUAAGGAGGCAACAACAUG | |
| F | GGUGAUACCAGCAUCGUCUUGAUGCCCUUGGCAGCACCC | 4 |
| | UGCUAAGGAGGUAACAACAUG | |
| G | GGUGAUACCAGCAUCGUCUUGAUGCCCUUGGCAGCACCC | 5 |
| | UGCUAAGGAGGUAACUUAAUG | |
| H | GGUGAUACCAGCAUCGUCUUGAUGCCCUUGGCAGCACCC | 6 |
| | UGCUAAGGAGGUGUGUUAAUG | |
| I | GGUGAUACCAGCAUCGUCUUGAUGCCCUUGGCAGCACCC | 7 |
| | UGCUAAGGAGGUCAACAAGAUG | |

In an embodiment the riboswitch has the sequence of Seq Id No: 2.

In an embodiment the target gene may be an endonuclease. There are many different types of endonuclease that can be used in various “genome-editing” strategies where they are engineered to cut specific genomic target sequences. These include Zinc Finger Nucleases (ZFN), Transcription Activator-like Effector Nucleases (TALEN) and Clustered Regularly Interspaced Short Palindromic Repeats (CRISPR) nucleases, homing meganucleases and standard restriction endonucleases (RE).

ZFNs include the Fok I endonuclease and an array of zinc finger binding domains that recognize the target DNA sequence. In TALENs, the zinc finger array is replaced by TAL effector repeats that guide targeting to the DNA. CRISPR/Cas9 genome editing requires a single guide (sg) RNA that directs the Cas9 endonuclease to a specific region of the genomic DNA, resulting in a DSB.

Homing meganucleases are part of selfish DNA elements, predominately introns (I-homing endonucleases) or encoded in-frame with a precursor protein as an intein (PI-homing endonucleases). They are characterised by their extreme specificity with target recognition sequences up to 40 bp in length. In this respect they differ from REs, whose target sequences consist of between 4 and 8 bp. The latter are of greatest utility in genome editing as the frequency of certain 8 bp recognition sequences in a genome can be extremely low, for instance, certain GC-rich 8 bp palindromic sequences can be entirely absent from AT-rich, clostridial genomes.

Homing meganucleases are divided into four families, characterized by common sequence motifs: LAGLIDADG, His-Cys-box, HN-H, and GIY-YIG. The former is the largest grouping. They contain two LAGLIDADG motifs and function as homodimers with one LAGLIDADG motif per polypeptide chain, e.g. I-CreI and I-MsoI, or as monomers with two motifs per polypeptide chain, e.g. PI-SceI, PI-PfuI, and I-DmoI.

In recent years genome editing strategies based on CRISPR/Cas (clustered regularly interspaced short palindromic repeats/CRISPR-associated proteins) have received particular prominence. They enable manipulation of the genome through the introduction of double-strand breaks in the DNA. In particular, the type II CRISPR-Cas9 system derived from Streptococcus pyogenes, has been the most extensively employed CRISPR system for genome editing purposes. In this system, the hybrid CRISPR RNA (crRNA):trans-activating crRNA (tracrRNA), or the simplified chimeric synthetic single guide RNA (sgRNA), combines with Cas9 to form a ribonucleoprotein complex. This complex then recognizes the target site, based on the protospacer adjacent motif (PAM) sequence, and induces a double strand break (DSB). In most bacteria, selection of mutant cells is achieved when the DNA editing template, which lacks the recognition site, is introduced into the genome via homologous recombination, enabling these mutant cells to “escape” from the cutting activity of Cas9.

Even though CRISPR/Cas9-based systems have been previously used in several Clostridium species, including C. pasteurianum, C. acetobutylicum, C. beijerinkii and C. difficile, this technology is still hindered by low transformation efficiencies, possibly related to the large size of the plasmid and the strong selection power of Cas9. The use of a Cas9 nickase (nCas9), a Cas9 mutant that introduces a breakage on only one strand of the chromosome, has been also demonstrated in Clostridium spp.; however, because nCas9 has a less powerful selection capacity than Cas9, the isolation of mutants becomes more time consuming, requiring several passages onto fresh medium.

The present invention provides for the first time the use of a synthetic riboswitch to control the expression of a DNA endonuclease, and in particular to control the expression of Cas9 expression, for use in CRISPR genome editing. More specifically, the invention provides the use of a synthetic riboswitch to control endonuclease, in particular Cas9, expression in the genus Clostridium.

The present invention provides the advantage that when unactivated the riboswitch prevents or reduces unwanted background levels of endonuclease activity in a cell. The present invention also has the advantage that when the riboswitch in activated by an inducer the endonuclease is expressed but the level of expression is low, it is sufficient to allow genome editing but not high enough to cause significant off target effects.

Also, CRISPR applications may be hampered by low transformation efficiencies. This may be because the expression of the nuclease before homologous recombination occurs leads to cell death and, as such, very few (sometimes none) colonies escape/survive from the activity of the nuclease. The use of an inducible expression control system that minimizes the expression of the nuclease until induced allows transformation efficiency to be improved. Furthermore, the small size of the riboswitch, around 89 nucleotides, does not add much to the construct size. This is in contrast to the incorporation of a transcription factor-based inducible system into a vector, which would generally add at least about 1.5 kb to an already large vector (i.e., cas9 is a very large gene, 4.2 kb). The size of the vector relates to the efficiency of transformation. A vector with cas9 under the control of a riboswitch will be smaller than a vector where cas9 is regulated via a transcription factor-based inducible system.

The endonuclease may be associated with CRISPR gene editing. The endonuclease may be Cas9, Cas9 nickase, dCas, Cpf1, C2c1, C2c2, C2c3, a Cas9 derivative, or any endonuclease suitable for use with CRISPR gene editing, or a homolog or functional variant thereof. The endoncuclease may be a Cas-derivative fused with a deaminase such as cytidine deaminase or adenine deaminase, the Cas-derivative may be any Cas9 effector protein such as Cas9 nickase or dCas9. Preferably the endonuclease is Cas9.

In an alternative embodiment the target gene may be an endonuclease characterised by a target recognition sequence of at least 8 bp. This includes, but is not restricted to, restriction enzymes such as the following (target sites are in brackets): AbsI (CCTCGAGG), SbfI/SdaI (CCTGCAGG), MreI (CGCCGGCG), MauBI (CGCGCGCG), SgrDI (CGTCGACG), Srfl (GCCCGGGC), AsiSI/SfaAI (GCGATCGC), Notl (GCGGCCGC), FseI (GGCCGGCC) and AcsI/SgsI (GGCGCGCC). Also included in this category are the meganucleases that have much larger recognition sites. These include, but are not restricted to, meganucleases such as the following (target sites are in brackets):—

AniI
(TTGAGGAGGTTTCTCTGTAAATAA);
I-CeuI
(TAACTATAACGGTCCTAAGGTAGCGA);
I-ChuI
(GAAGGTTTGGCACCTCGATGTCGGCTCATC);
I-CpaI
(CGATCCTAAGGTAGCGAAATTCA);
I-CpaII
(CCCGGCTAACTCTGTGCCAG);
I-CreI
(CTGGGTTCAAAACGTCGTGAGACAGTTTGG);
I-DmoI
(ATGCCTTGCCGGGTAAGTTCCGGCGCGCAT);
H-DreI
(CAAAACGTCGTAAGTTCCGGCGCG);
I-HmuI
(AGTAATGAGCCTAACGCTCAGCAA);
I-HmuII
(AGTAATGAGCCTAACGCTCAACAA);
I-LlaI
(CACATCCATAACCATATCATTTTT);
I-MsoI
(CTGGGTTCAAAACGTCGTGAGACAGTTTGG);
PI-PfuI
(GAAGATGGGAGGAGGGACCGGACTCAACTT);
PI-PkoII
(CAGTACTACGGTTAC);
I-PorI
(GCGAGCCCGTAAGGGTGTGTACGGG);
I-PpoI
(TAACTATGACTCTCTTAAGGTAGCCAAAT);
PI-PspI
(TGGCAAACAGCTATTATGGGTATTATGGGT);
I-ScaI
(TGTCACATTGAGGTGCACTAGTTATTAC);
I-SceI
(AGTTACGCTAGGGATAACAGGGTAATATAG);
PI-SceI
(ATCTATGTCGGGTGCGGAGAAAGAGGTAATGAAATGGCA);
I-SceII
(TTTTGATTCTTTGGTCACCCTGAAGTATA);
I-SecIII
(ATTGGAGGTTTTGGTAACTATTTATTACC);
I-SceIV
(TCTTTTCTCTTGATTAGCCCTAATCTACG);
I-SceV
(AATAATTTTCTTCTTAGTAATGCC);
I-SceVI
(GTTATTTAATGTTTTAGTAGTTGG);
I-SceVII
(TGTCACATTGAGGTGCACTAGTTATTAC);
I-Ssp6803I
(GTCGGGCTCATAACCCGAA);
I-TevI
(AGTGGTATCAACGCTCAGTAGATG);
I-TevII
(GCTTATGAGTATGAAGTGAACACGTTATTC);
I-TevIII
(TATGTATCTTTTGCGTGTACCTTTAACTTC);
PI-TliI
(TAYGCNGAYACNGACGGYTTYT);
PI-TliII
(AAATTGCTTGCAAACAGCTATTACGGCTAT);
I-Tsp061I
(CTTCAGTATGCCCCGAAAC);
I-Vdi141I
(CCTGACTCTCTTAAGGTAGCCAAA).

In common with Cas9, the regulated expression of any of these enzymes may be used to introduce DSBs into the genome of the target cell wherever a recognition site is present. For example, various recombination strategies have been devised based on the regulated production of I-SceI in a cell in which its target site has been introduced into the genome. In its simplest form, a plasmid carrying a mutant allele is integrated into the genome via homologous recombination between a flanking left homology arm (LHA) and a right homology arm (RHA)—a knock-out (KO) cassette—together with an I-SceI restriction site that resides outside of the KO cassette. Regulated production of I-SceI, using an inducible promoter system such as the riboswitch, will result in the cleavage of the genome of all of those cells which carry the integrated plasmid, together with the I-SceI recognition site, leading to cell death. Those plasmids in which the plasmid has excised, as a consequence of homologous recombination between the duplicated LHA or RHA, will lack a I-SceI site and survive. Dependant on which homology arm mediates recombination, the surviving cells will carry either a wildtype or mutant allele. Other, more sophisticated variations of this approach exist, where the use of I-SceI or similar is combined with the use of lambda-Red technology etc, but the principle remains the same, ie., regulated production of I-SceI leads to elimination of the unwanted cells, and the enrichment/selection of the desired bioengineered variants. This approach may be taken with any meganuclease or restriction endonuclease, not just I-SceI.

In an alternative embodiment the target gene may be a sigma factor. Sigma (σ) factors control the promoter selectivity of bacterial RNA polymerase (RNAP). On binding to RNAP, σ factors allow efficient promoter recognition and transcription initiation. Aside from promoter recognition, they contribute to DNA strand separation, and then dissociate from the core enzyme following transcription initiation. Procaryotes produce a number of different σ factors, each of which recognises a specific promoter sequence. In this way, the production of one particular sigma factor can simultaneously regulate the expression of discrete sets of genes which are under the control of the target promoter sequence. Sigma factors are classified into two structurally unrelated families, the σ⁷⁰ and the σ⁵⁴ families. The σ⁷⁰ family includes primary sigma factors responsible for the expression of housekeeping genes (e.g., σ^{A} in Bacillus subtilis) as well as related alternative sigma factors; σ54 forms a distinct subfamily of sigma factors referred to as σ^{N}. The number of genes can vary dependent on the sigma factor. The expression of most genes in a bacterial cell is dependent on the expression of the ‘housekeeping’ sigma factor σ⁷⁰, but bacteria can express different sigma factors in response to different environmental conditions.

Alternative sigma factors can be responsible for the expression of a small subset of genes, which can be extremely limited. One such class of sigma factor are those responsible for the expression of the large, clostridial extracellular virulence factors of pathogenic strains of Clostridium botulinum, Clostridium tetani and Clostridium difficile, and a bacteriocin by Clostridium perfringens. These particular sigma factors have been assigned to σ⁷⁰ group 5 (Dupuy and Matamouros, 2006, Research Microbiology, 157: 201-205) and recognise highly specific promoter elements which uniquely precede the toxin/bacteriocin genes of these bacteria. No other genes in the genome are known to be under the transcriptional control of these sigma factors. As the large size of these toxins/bacteriocins places an appreciable metabolic burden on the cell, it is important that their production is not constitutive but limited to specific conditions when they are required, ie., in the case of C. difficile when growing in the GI tract. Accordingly, their expression needs to be tightly regulated. This is achieved by making gene transcription absolutely reliant on a specific σ factor, and tightly regulating production of the σ factor.

The group 5 RNA polymerase sigma factor may be TcdR (from Clostridium difficile), BotR (from Clostridium botulinum), TetR (from Clostridium tetani) or UviA (from Clostridium perfringens). Preferably, the group 5 RNA polymerase sigma factor is BotR. Preferably, the group 5 RNA polymerase sigma factor is TetR. Preferably, the group 5 RNA polymerase sigma factor is TcdR. Preferably, the group 5 RNA polymerase sigma factor is UviA. The group 5 RNA polymerase sigma factor may have a sequence identity or sequence homology of at least 60%, 65%, 70%, 75%, 80%, 85%, 90%, 95%, 98%, 99% of, or is identical to, one or more of BotR, TetR, TcdR or UviA.

Due to the specificity of this family of σ⁷⁰ factors, expression of either native or heterologous genes preceded by the requisite target promoter are absolutely reliant on availability of the σ factor. For example expression of the various genes encoding the components of the Clostridium botulinum toxin complex, is reliant on the presence of BotR, as will be the expression of any heterologous gene placed under the transcriptional control of the two promoter targets, those that precede either the ntnh (Pₙₜₙₕ) or the ha70 (Pₕₐ₇₀) gene. It follows that if σ factor expression (BotR) is placed under the control of the riboswitch, then effectively there will be no transcription from their target promoters (Pₙₜₙₕ or Pₕₐ₇₀), and consequent production of the encoded products of the downstream genes, in the absence of inducer. This tight level of repression may be advantageous from a safety perspective as it will limit the production of potentially highly toxic molecules, such as botulinum toxin, to those circumstances where such production is required, eg., in the commercial production of toxin for cosmetic or therapeutic purposes.

As a consequence of the low dynamic range of induction of the genetic construct of the invention, if the target gene is BotR, or a similar σ factor, production of BotR or the similar σ factor may be relatively low upon activation of the riboswitch. However, because each σ factor molecule brings about multiple transcriptional events at their target promoter (Pₙₜₙₕ or Pₕₐ₇₀, in the case of BotR) the relative level of induction may be amplified. Thus, the level of expression of the gene concerned may be higher than if the promoters concerned (Pₙₜₙₕ or Pₕₐ₇₀, in the case of BotR) were place directly under the control of the riboswitch.

The genetic construct may comprise a) a promoter suitable for use in a prokaryotic host, b) a riboswitch to regulate translation, and c) the coding region for a target gene.

The genetic construct of the invention may be introduced into a host cell by using any suitable means, such as endocytic uptake, microinjection, ballistic bombardment, a particle gun, electroporation, transduction, transfection, infection or cell fusion. Preferably, the genetic construct is introduced into the cell by using a vector.

Thus, according to another aspect of the invention, there is provided a vector comprising the genetic construct of the invention.

The vector may be a recombinant vector. The vector may be a virus, a virus-like particle, a plasmid, a cosmid, a phage, a transposon or a liposome.

According to another aspect of the invention, there is provided a host cell comprising the genetic construct according to the invention or a vector according to the invention.

The host cell may be a bacterium, a plant, an algae, a fungi or a protozoa. Preferably, the cell is a bacterium.

The bacterium may be a Gram positive bacterium or Gram negative bacteria. The bacteria may be of the genus Bacillus or Clostridium. The bacterium may be Clostridium sporogenes.

The bacterial cell may be any bacterial species, but preferably members of the bacterial phylum Firmicutes composed of the class Clostridia (orders Clostridiales, Halanaerobiales, Natranaerobiales and The rmoanaerobacterales), the class Bacilli (orders Bacillales and Lactobacillales) and the class Mollicutes (orders Acholeplasmatales, Anaeroplasmatales, Entomoplasmatales, Haloplasmatales and Mycoplasmatales).

The bacterium may be within the order of Clostridiales, Halanaerobiales, Natranaerobiales, Thermoanaerobacterales, Bacillales, Lactobacillales, Acholeplasmatales, Anaeroplasmatales, Entomoplasmatales, Haloplasmatales or Mycoplasmatales. Preferably, the bacterium is within the order of Clostridiales.

Within the order Clostridiales is the genus, Clostridium. Preferred species are C. aceticum, C. acetobutylicum, C. aerotolerans, C. baratii, C. beijerinckii, C. bifermentans, C. botulinum, C. butyricum, C. cadaveris, C. cellulolyticum, C. chauvoei, C. clostridioforme, C. colicanis, C. difficile, C. drakei C. estertheticum, C. fallax, C. feseri, C. formicaceticum, C. glycolicum, C. histolyticum, C. innocuum, C. kluyveri, C. ljungdahlii, C. lavalense, C. magnum. C. mayombei, C. methoxybenzovorans, C. novyi, C. oedematiens, C. paraputrificum, C. pasteurianum, C. perfringens, C. phytofermentans, C. piliforme, C. ragsdalei, C. ramosum, C. scatologenes, C. septicum, C. sordellii, C. sporogenes, C. sticklandii, C. tertium, C. tetani, C. thermocellum, C. thermosaccharolyticum, C. tyrobutyricum, C. paprosolvens, C. saccharobutylicum, C. carboxidovorans, C. scindens C. autoethanogenum, C. diolis, C. aurantibutyricum, C. felsineum, C. puniceum, C. roseum, C. saccharoperbutylacetonicum, C. tetanomorphum, and Clostridioides difficile, as well as other acetogenic anaerobes, such as, Acetobacterium woodii, Acetonema longum, Alkalibaculum bacchi, Blautia producta, Butyribacterium methylotrophicum, Eubacterium limosum, Oxobacter pfennigii, Moorella thermoacetica, Moorella thermoautotrophica, Thermoanaerobacter kiuvi.

Within the order Bacillales are Bacillaceae which include the genera Bacillus and Geobacillus, and Staphylococcaceae, which include the genus Staphylococcus. Preferred Bacillus species are: B. alcalophilus, B. aminovorans, B. amyloliquefaciens, B. anthracis, B. caldolyticus, B. circulans, B. coagulans, B. cereus, B. globigii, B. licheniformis, B. natto, B. polymyxa, B. phaericus, B. stearothermophilus, B. smithii, B. subtilis, B. thermoglucosidasius, B. thuringiensis and B. vulgatis. Preferred Geobacillus species are: G. debilis, G. stearothermophilus, G. thermocatenulatus, G. thermoleovorans, G. kaustophilus, G. thermoglucosidasius, G. thermodenitrificans, G. gargensis, G. jurassicus, G. lituanicus, G. pallidus, G. subterraneus, G. tepidamans, G. thermodenitrificans, G. thermoglucosidasius, G. thermoleovorans, G. toebii, G. uzenensis and G. vulcani. Preferred Staphylococcus species include: S. arlettae, S. aureus, S. auricularis, S. capitis, S. caprae, S. carnosus, S. chromogenes, S. cohnii, S. condiments, S. delphini, S. devriesei, S. epidermidis, S. equorum, S. fells. S. jleurettii, S. gallinarum, S. haemolyticus, S. hominis, S. hyicus, S. intermedius, S. kloosii, S. leei, S. lentus, S. lugdunensis, S. lutrae, S. lyticans, S. massiliensis, S. microti, S. muscae, S. nepalensis, S. pasteuri, S. pettenkoferi, S. piscsfermentans, S. pseudintermedius, S. pulvereri, S. rostri, S. saccharolyticus, S. saprophyticus, S. schleiferi, S. sciuri, S. simiae, S. simulans, S. stepanovicii, S. succinus, S. vitulinus, S. warneri and S. xylosus.

The bacterial cell may be C. acetobutylicum, C. difficile, C. beijerinckii, C. ljungdahlii, C. kluyveri, C. botulinum, C. beijerinckii, C. autoethanogenum, C. pasteurianum, C. saccharobutylicum, C. carboxidovorans, C. cellulovorans, C. sporogenes, C. phytofermentans, C. ragsdalei, C. tyrobutyricum, C. perfringens, C. butyricum, C. cellulolyticum, C. formicaceticum, C. novyi, C. scatologenes, C. septicum, C. sordellii, C. sticklandii, C. tetani, C. thermocellum, C. thermosaccharolyticum, C. paprosolvens, C. scindens, or C. bifermentans.

Preferably, the bacterial cell is a species selected from the group consisting of C. acetobutylicum, C. aerotolerans, C. autoethanogenum, C. baratii, C. beijerinckii, C. bifermentans, C. botulinum, C. butyricum, C. cadaveris, C. cellulolyticum, C. cellulovorans, C. chauvoei, C. clostridioforme, C. colicanis, C. difficile (now renamed Clostridioides difficile), C. estertheticum, C. fallax, C. feseri, C. formicaceticum, C. histolyticum, C. innocuum, C. kluyveri, C. ljungdahlii, C. lavalense, C. novyi, C. oedematiens, C. paraputrificum, C. pasteurianum, C. perfringens, C. phytofermentans, C. piliforme, C. ragsdalei, C. ramosum, C. roseum, C. saccharoperbutylacetonicum, C. scatologenes, C. septicum, C. sordellii, C. sporogenes, C. sticklandii, C. tertium, C. tetani, C. thermocellum, C. thermosaccharolyticum, C. tyrobutyricum, C. paprosolvens, C. saccharobutylicum, C. carboxidovorans, C. scindens, or C. autoethanogenum.

The bacterial cell may be C. phytofermentans, C. hylemonae, C. leptum, C. symbiosum, C. nexile, C. ramosum, C. bolteae, C. asparagiforme, C. methylpentosum, C. butyricum, C. sporogenes and C. scindens.

The bacterial cell may be Cupriavidus necator or metalodurans or is a cyanobacteria

In a preferred embodiment the host cell is a Clostridium cell. One particular challenge with Clostridium is to identify a workable inducible expression system. Clostridium is a large genus of Gram-positive, anaerobic, spore-forming bacteria that includes representatives relevant to both human and animal diseases as well as to the industrial production of chemicals and fuels. Whilst the majority of these species are studied for independent purposes, the emerging field of synthetic biology brings them all together under the same scope—the engineering of novel strains with new functionalities. These designated novel strains are on the one hand facilitating the study of fundamental biological processes and on the other hand, they are advancing biotechnological applications. Such applications include the production of platform chemicals and biofuels (e.g., Clostridium pasteurianum, Clostridium acetobutylicum); cellulosic and hemicellulosic biomass degradation (e.g., Clostridium celluloliticum); carbon fixation (e.g., Clostridium ljungdahlii and Clostridium autoethanogenum) and anti-cancer therapeutics (e.g., Clostridium sporogenes). Only a few studies regarding the use of inducible expression systems in Clostridium have been reported. These include a lactose-inducible system (LAC) from Clostridium perfringens, an arabinose-inducible system (ARA), based on the ARAi regulon from C. acetobutylicum, and the tetracycline-inducible system (TET), originally adapted from E. coli. Despite evidence of a dose-dependent induction, both, the LAC and ARA systems are hampered by a significant level of gene expression in the absence of the inducer, compromising their dynamic range and making them unsuitable for applications where a tight control of gene expression is needed. On the other hand, the TET system exhibits very low basal expression and has the highest inducing efficiency among the reported inducible promoters applied into Clostridium spp. thus far. However, optimal working conditions of the TET system, require high doses of the inducer, but elevated concentrations of the tetracycline-analogue, anhydrotetracycline, demonstrated significant inhibitory effects on cell growth.

Another important limitation that Clostridium spp. faces towards their potential application in synthetic biology projects, is the lack of fast and reliable methods for chromosomal manipulation. Traditionally, chromosomal modifications have been primarily achieved via insertional mutagenesis using ClosTron (Heap, J. T. et al. J. Microbiol. Methods 80, 49-55 (2010)) or via a special form of allelic exchange termed allele-coupled exchange (ACE) (Heap, J. T. et al. Nucleic Acids Res. 40, e59 (2012)); unfortunately, both methods are far from ideal. In ClosTron, for example, the end product is not a true deletion of the gene but rather an interruption of the gene's function which may also lead to polar effects on downstream genes. On the other hand, although ACE allows a more precise modification of the genome, it is lengthy and relies on a counter selection marker (such as pyrE, which may not always be available).

The genetic construct of the invention addresses many of the problems currently faced with respect to Clostridium spp.

According to a further aspect the invention provides a kit for regulating expression of a target gene, wherein the kit comprises the genetic construct of the invention. If the target gene is an endonuclease for use in CRISPR gene editing the kit may further comprise a sequence-specific guide RNA.

According to a further aspect the invention provides a method of controlling expression of a target gene in a cell comprising:
i) transforming a host cell with a genetic construct, the construct comprising polynucleotide to be transcribed, wherein the polynucleotide comprise is a coding region encoding a target gene operably linked to a riboswitch;
ii) exposing the transformed cell to an inducer of the riboswitch thereby effecting expression of the target gene.

If the target gene is an endonuclease the method may further comprise transforming the cell with gRNAs needed to target the endonuclease activity, or using a cell which already contains the gRNAs needed to target the endonuclease activity.

According to a yet further aspect the invention provides a method of controlling expression of a target gene in a cell comprising:
i) providing a host cell which contained a genetic construct, the construct comprising polynucleotide to be transcribed, wherein the polynucleotide comprise is a coding region encoding a target gene operably linked to a riboswitch;
ii) exposing the transformed cell to an inducer of the riboswitch thereby effecting expression of the target gene.

Where the target gene is an endonuclease the cell may further comprise gRNAs needed to target the endonuclease activity.

All of the features described herein (including the accompanying claims, abstract and drawings) and/or all of the steps of any method or process so disclosed, may be combined with any of the above aspects in any combinations, except combinations where at least some of such features and/or steps are mutually exclusive.

For a better understanding of the invention, and to show how embodiments of the same may be carried into effect, reference will now be made, by way of example, to the accompanying Figures, in which:—

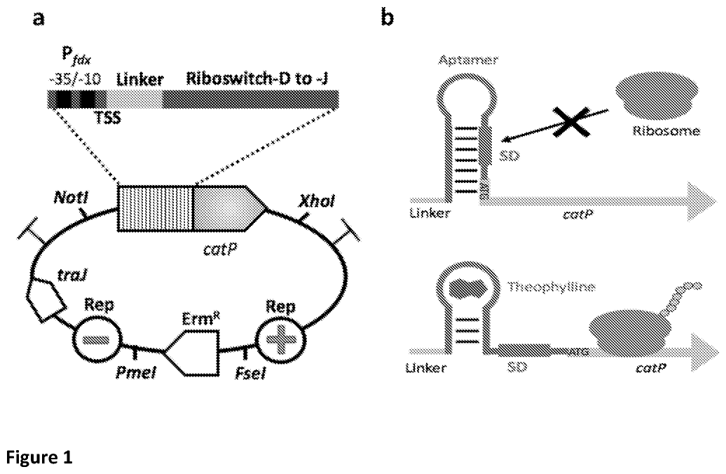

FIG. 1—FIG. 1a shows Escherichia coli (E. coli)—Clostridium shuttle plasmid pMTL-IC101 containing the catP reporter under the control of the ferredoxin promoter. Riboswitches-D to -J were placed downstream of the TSS. (−) is the Gram-negative ColE1 RNA II origin of replication that allows replication of the shuttle plasmid in E. coli. traJ encodes the TraJ protein, needed for conjugal transfer. (+) is the pBP1 replicon of the Gram-positive Clostridium botulinum. FIG. 1b is a schematic representation of a functional model of the theophylline responsive riboswitch. In the absence of theophylline, the riboswitch forms a stem-loop structure that sequesters the ribosome binding site (RBS) in the mRNA transcript. When theophylline binds to the aptamer, the riboswitch conformation changes resulting in the release of the RBS and initiation of translation of the gene of interest (catP).

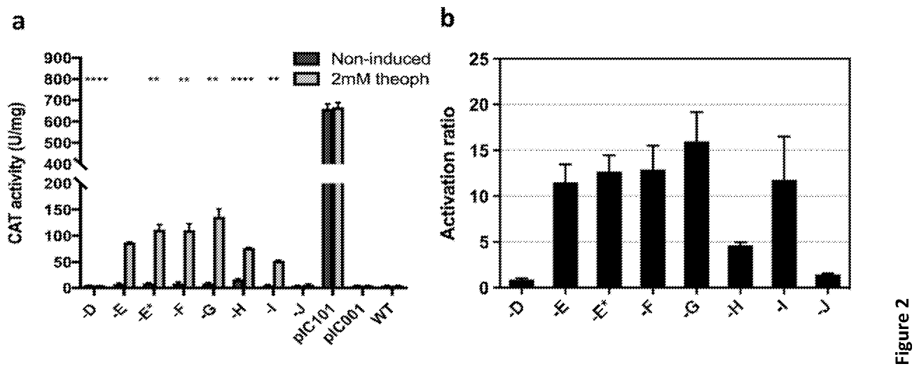

FIG. 2—FIG. 2a shows CAT activity and its ligand-dependant induction in each pMTL-IC111-reporter plasmid. Pre-cultures were inoculated into fresh TYG medium to a final OD600=0.5; each culture was split into two equal volumes, and one of them was induced with 2 mM theophylline for 4 hours. The reporter plasmids pMTL-IC101 (Pfdx-catP), pMTL-IC001 (promoterless catP) as well as the WT strain were used as controls. Error bars represent standard deviations of three biological replicates. Asterisks indicate statistically significant induction values for *p≤0.0332, **p≤0.0021, ***p≤0.0002, ****p≤0.0001 (paired two-tailed Student's t-test). FIG. 2b shows the activation ratio of riboswitches-D to -J. The activation ratio in each riboswitch-based reporter plasmid was calculated by dividing the value of CAT activity measured in the presence of the inducer by the value of that in the absence of inducer.

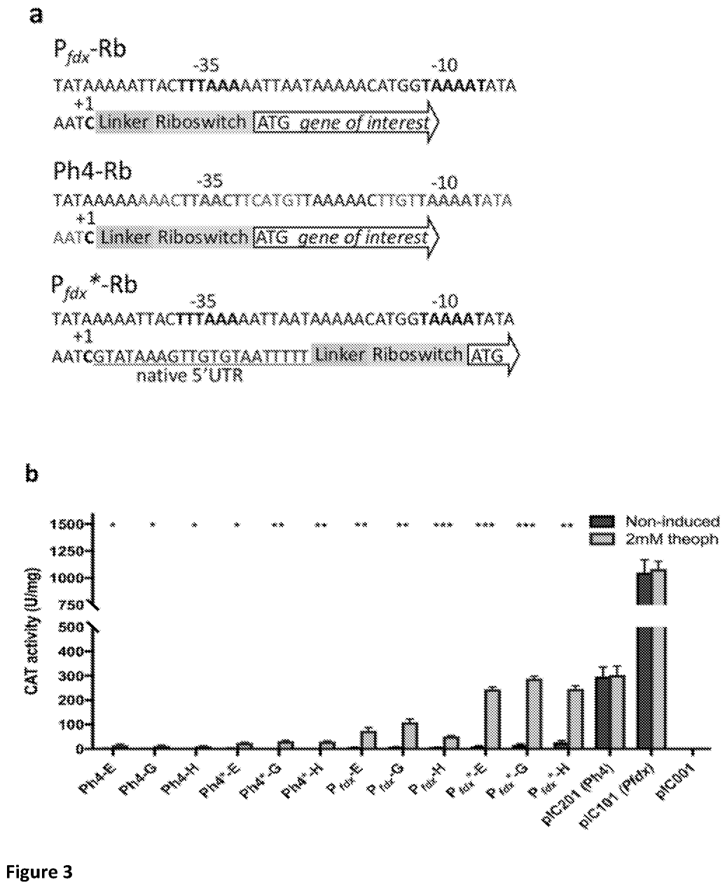

FIG. 3—FIG. 3a shows the sequences of the constructed theophylline-responsive switches. The predicted −35 and −10 sequences are in bold. The experimentally determined TSS via 5′RACE (Fig. S2) are indicated with +1. The 5′UTR sequences downstream of the TSS are underlined. Core elements replaced to create the synthetic hybrid promoter Ph4 are in red. (b) Library of theophylline-dependent riboswitches tested in Clostridium sporogenes in the presence and absence of the inducer. The reporter plasmids pMTL-IC201 (Ph4-catP), pMTL-IC101 (Pfdx-catP) and pMTL-IC001 (promoterless catP) were used as controls. Error bars represent standard deviations of three biological replicates. Asterisks indicate statistically significant induction values (paired two-tailed Student's t-test).

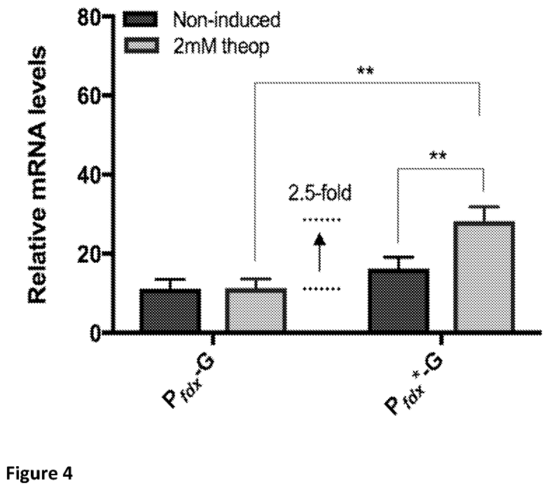

FIG. 4—illustrates the transcript abundance determined by RT-qPCR relative to two reference genes, 16Srrn and gyrA. Total RNA was isolated from late exponential cultures grown in the presence and absence of 2 mM theophylline. Error bars represent standard deviations of three biological replicates. Asterisks indicate statistically significant induction values (paired/unpaired two-tailed Student's t-test).

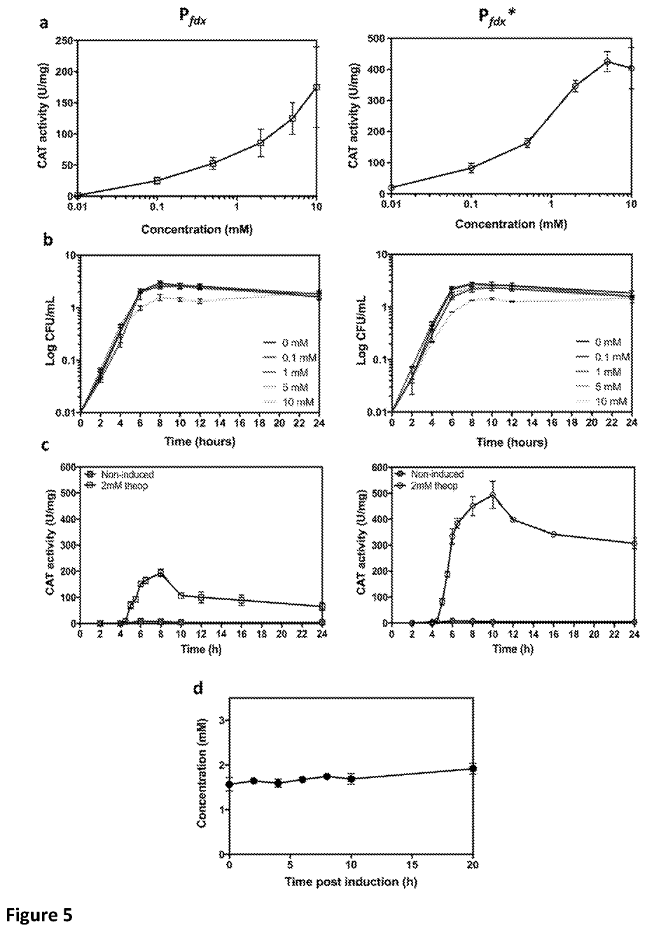

FIG. 5—shows dynamic and kinetic profiles of the theophylline responsive riboswitch located downstream of Pfdx or Pfdx*. FIG. 5a shows the response to different concentrations of the inducer theophylline. Cells containing the reporter plasmid with riboswitch G downstream of either Pfdx or Pfdx* were cultivated in TYG medium supplemented with various concentrations of theophylline (0, 0.1, 0.5, 2, 5 and 10 mM) at early exponential growth phase (4 hours of growth, OD600≈0.5); CAT activity was measured on cell lysates from stationary cultures. FIG. 5b shows the optical density (OD600) of C. sporogenes harbouring the same reporter plasmids in response to 0.1-10 mM of theophylline supplemented at time zero. FIG. 5c shows CAT expression profiles over time. Cells harbouring the reporter plasmids were cultivated in TYG media in the absence or presence of 2 mM theophylline; CAT was measured on cell lysates from stationary cultures. FIG. 5d shows the stability profile of theophylline in C. sporogenes. In all cases, error bars represent the standard deviations of three biological replicates.

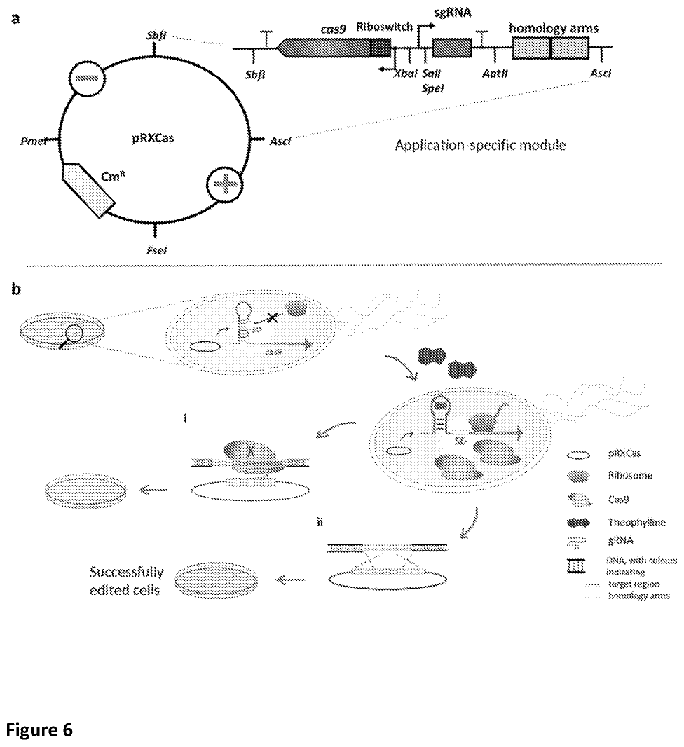

FIG. 6—FIG. 6a is a schematic illustration of the RiboCas vector. It contains the four unique restriction sites AscI, FseI, PmeI and SbfI which are used for modular assembly. (−) is the Gram-negative origin of replication that allows replication of the shuttle plasmid in E. coli (+) is the Gram-positive replicon. The application-specific module harbours the components of the editing tool, including the gene encoding Cas9, the sgRNA and the homologous DNA template needed for homologous recombination. The terminators CD0164 (derived from C. difficile) and Tfdx (derived from C. pasteurianum) are placed downstream of cas9 and the sgRNA respectively. The system was designed to be compatible with our previous pMTL80000 vector series enabling rapid exchange of selection markers and origins of replication. FIG. 6b is an illustration of the RiboCas-mediated genome editing. Transformed cells survive on selective media in the absence of theophylline due to the tight repression exerted by the riboswitch, which impedes the translation of the nuclease Cas9. After induction, the translated Cas9 forms a complex with the sgRNA (Cas9-sgRNA) inducing a double-strand breakage on the target DNA. (i) The Cas9-sgRNA complex is lethal to the unedited cells, which are killed during the process. (ii) Cells are able to survive only if homologous recombination occurs between the gene-targeting plasmid and the genome.

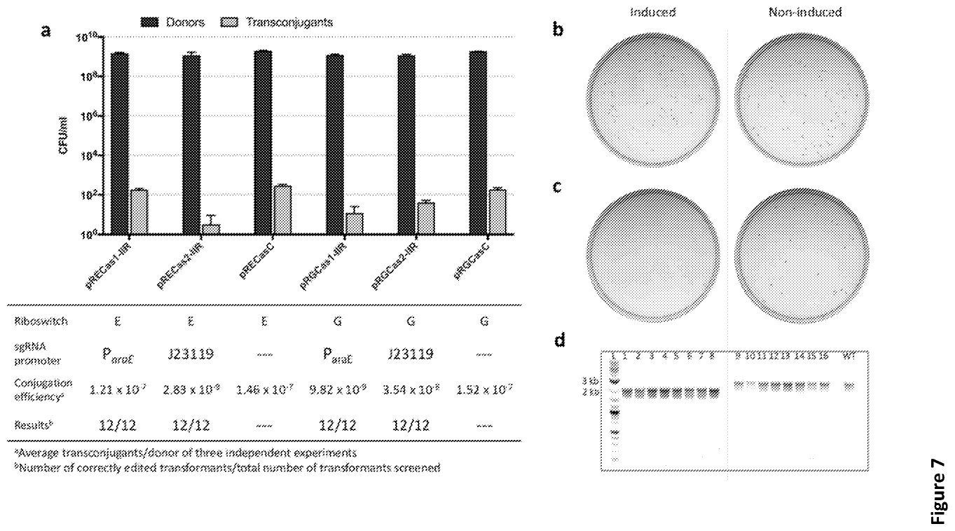

FIG. 7—FIG. 7a shows the conjugation efficiency of different RiboCas plasmids involved in CRISPR-mediated gene editing. Error bars represent standard deviations of three biological replicates on three independent experiments. The data summarize the obtained editing results from each RiboCas plasmid. FIG. 7b shows the CFUs obtained from conjugation of pRECas1C (RiboCas vector lacking both homology arms and sgRNA) on selective media in the presence and absence of the inducer theophylline. FIG. 7c shows the CFUs obtained from conjugation of pRECas1G-IIR (RiboCas vector lacking the homology arms) on selective media in the presence and absence of theophylline. FIG. 7d shows confirmation of the gene deletion using colony PCR. Lane L, the 2-log DNA marker with sizes (kbp) on the left; lanes 1-8, colony PCR on colonies from selective plates containing theophylline; lanes 9-16, colony PCR on colonies from selective plates lacking theophylline; WT (wildtype).

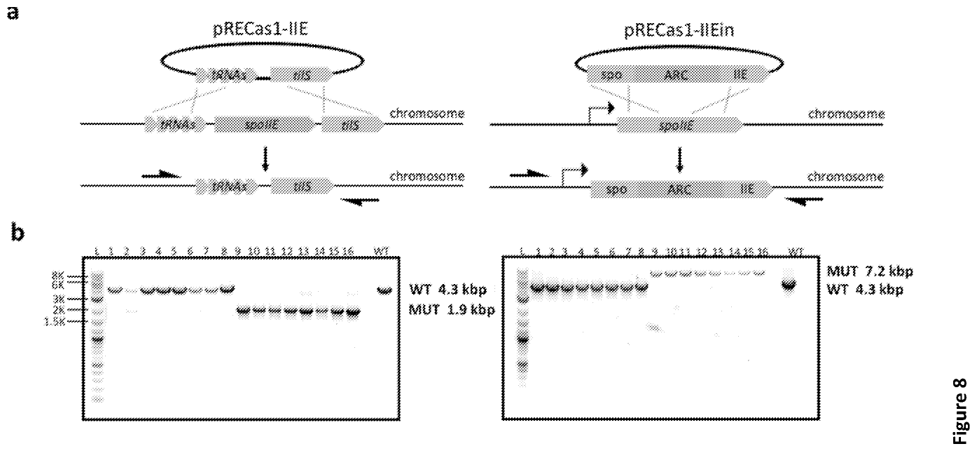

FIG. 8—FIG. 8a is a schematic representation of the deletion of spoIIE (via pRECas1_IIE) and the integration of an ARC in the spoIIE locus (via pRECas1_IIEin) in the chromosome of C. sporogenes. The ARC codifies the gene ermB that confers resistance to the antibiotic erythromycin. After integrated in the chromosome, the gene can be expressed under the control of the spoIIE promoter, enabling the selection of colonies in media containing erythromycin. FIG. 8b shows PCR screening of the spoIIE deletion and integration of the ARC. The 1.9, 7.2 and 4.3 kbp bands represent the spoIIE deletion, ARC integration and WT genotypes. Lane L, 2-log DNA marker with sizes (kbp) on the left; lanes 1-8 uninduced colonies; lanes 9-16 induced colonies.

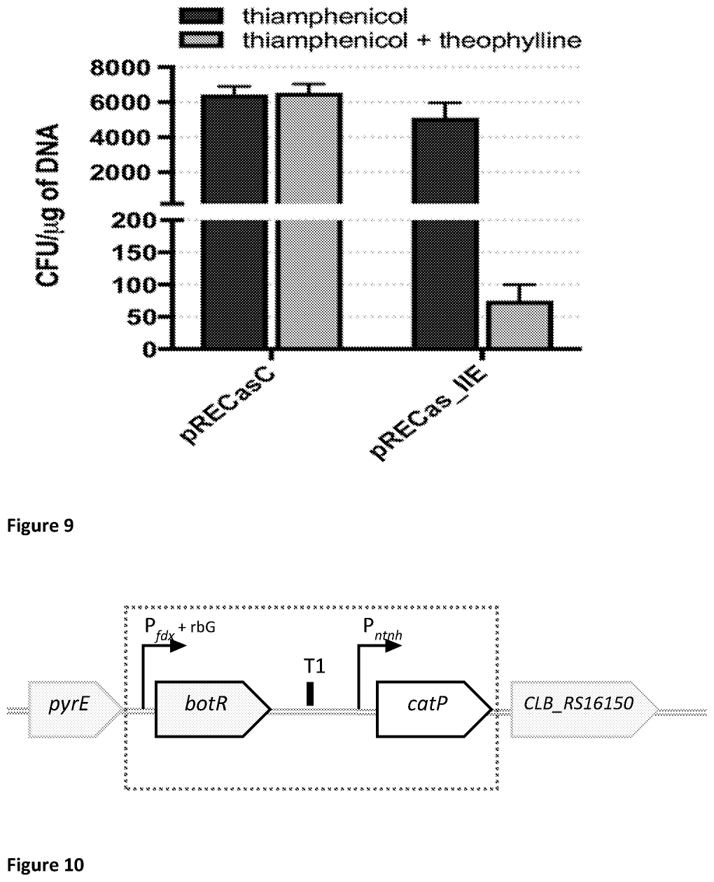

FIG. 9 shows the transformation efficiencies of RiboCas plasmids in C. pasteurianum, without (pRECasC) or with (pRECas_IIE) the spoIIE-targeting sgRNA and the DNA editing template, when plated on selective media in the absence and presence of the inducer theophylline.

FIG. 10 is a schematic diagram of a riboswitch-controlled gene expression circuit, whereby botR is the target gene. The circuit is integrated in the pyrE locus of C. botulinum ATCC 19397 ΔClA. Pfdx is the promoter upstream of the ferredoxin gene from C. sporogenes NCIMB 10696; rbG is a theophylline-responsive riboswitch controlling translation (SEQ ID no. 5); botR is the gene encoding the alternative sigma factor BotR from C. botulinum ATCC 19397; T1 is a terminator sequence downstream of the FAD-oxidoreductase gene from C. tetani; Pₙₜₙₕ is the upstream regulatory region of the non-toxic non-haemagglutinin component of the neurotoxin complex from C. botulinum ATCC 19397 (controlled by BotR); catP is the gene encoding the chloramphenicol acetyltransferase reporter (CAT; EC: 2.3.1.28); CLB_RS16150 is the gene downstream of the integrated module in the genome of C. botulinum ATCC 19397.

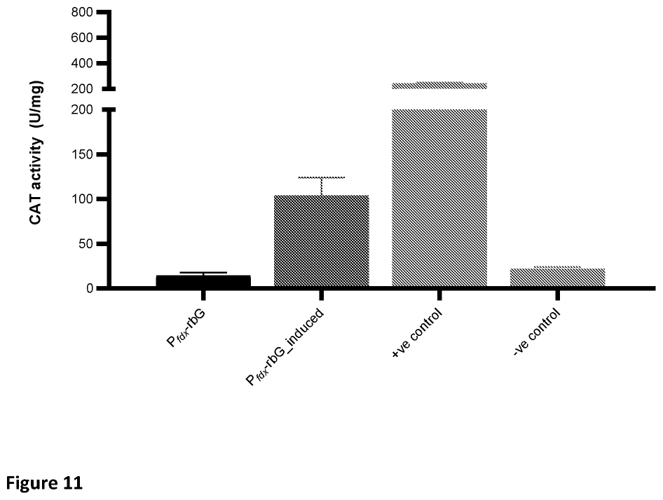

FIG. 11 CAT assay results. The strain with P_{fdx}-rbG-botR-T1-Pₙₜₙₕ-catP integrated in the pyrE locus was grown in TYG medium and induction with 2 mM theophylline took place at the early exponential growth phase (OD600≈0.5). CAT activity in the uninduced state is represented by a black bar and CAT activity in the induced state is represented by a red bar. The +ve control is a strain with P_{fdx}-catP integrated in the same locus and the -ve control is the WT strain. Error bars represent standard deviations of three biological replicates.

### Materials and Methods

### Strains, Media and Growth Conditions.

All the E. coli and Clostridium strains used in this study are listed in Table 2. E. coli TOP 10 (Invitrogen) was used as a general host for plasmid construction and propagation. E. coli CA434 was used as the donor strain for conjugation. Plasmid DNA for the transformation of C. pasteurianum was methylated in vivo by propagation in the E. coli host CR1, which harbours the plasmid pCR1, encoding the M.BepI methyltransferase, as previously described (Schwarz, K. M. et al. Metab. Eng. 40, 124-137 (2017)). All E. coli strains were transformed through electroporation using a MicroPulser™ system (BioRad). E. coli strains were grown at 30 or 37° C., in Luria-Bertani (LB) medium supplemented with chloramphenicol (25 μg/mL in solid and 12.5 μg/mL in liquid media), erythromycin (500 μg/mL) or kanamycin (50 μg/mL) when necessary. Growth media for the different Clostridium species are specified on Table 3. Media for clostridial strains were supplemented with the following antibiotic/inducer/supplement when appropriate: thiamphenicol (15 μg/mL), erythromycin (10 μg/mL), cefotoxin (16 μg/mL), D-cycloserine (500 μg/mL), theophylline (0.1-10 mM), glucose 0.05% w/v. Clostridium strains were grown at 37° C. in an anaerobic cabinet (MG1000 anaerobic workstation; Don Whitley Scientific Ltd).

### Reagents.

All PCR reactions were performed using KOD-Hot Start Polymerase 2× Master Mix (Merck Millipore) or DreamTaq Green PCR Master Mix (Thermo Fisher Scientific). T4 ligase (Promega) was used for DNA ligation reactions. Restriction enzymes were purchased from New England Biolabs. Theophylline was purchased from Sigma-Aldrich.

### Plasmid Design, Construction and Transformation.

Oligonucleotide primers were synthesized by Sigma-Aldrich and are listed in Table 4. Plasmids were constructed by restriction enzyme-based cloning procedures. Constructs were verified by DNA sequencing (Eurofins). All the plasmids used in this study are listed in Table 5. Details of plasmid construction are given in the Supporting Information. Protospacer sequences were designed according to the protocol described at http://benchling.com/pub/ellis-crspr-tools.

### Growth and CAT Activity Assays.

For each tested system, three independently conjugated C. sporogenes cultures were grown for 12 hours with selection before being diluted to a starting OD₆₀₀ of 0.01 in fresh medium. For evaluation of CAT activity at a single time point, cultures were induced at an OD₆₀₀≈0.5 with 2 mM theophylline; cultures were collected at stationary phase 4 hours after induction. For CAT expression assays over time, cultures were prepared as for the single time point measurements, collecting the samples at the specific data points. For dose-dependency assays, cultures were induced with increasing concentrations of theophylline (0-10 mM). In all cases, after sample collection, pellets were obtained and stored at −20° C. until CAT activity was determined.

CAT activity was measured on cell lysates according to the method of Shaw Methods Enzymol. 43, 737-55 (1975). Cell lysates were obtained using BugBuster Master Mix lysis buffer (Novagen), according to the manufacturer's protocol. 150 μL of a master mix consisting of 94 mM Tris buffer (pH 7.8), 0.19 mM acetyl Coenzyme A, 0.0833 mM DTNB (5,5′-dithiobis-2-nitrobenzoic acid) and 0.005% (w/v) chloramphenicol, were injected into each well of a 96-well clear-bottom plate (Greiner Bio One International) containing 10 μL of cell lysates. Absorbance was measured at 412 nm for 1 min using a CLARIOstar plate reader (MBG Labtech GmbH), set at 25° C. The rate of increase of absorption was used to calculate CAT activity (U/ml) using the following equation, where 0.2 is the total volume (in mL) of assay, df is the dilution factor, 0.0136 is the micromolar extinction coefficient for DTNB at 412 nm and 0.01 is the volume of cell lysate used.

$$Units / ml CAT = \frac{( Δ {A}_{412} / min test - Δ {A}_{412} / min blank ) ( 0.2 ) ( d f ) )}{( 0.013 \; 6 ) ( 0.01 )} \qquad$$

CAT activity was further normalized by the total protein concentrations obtained using a BCA assay (Thermo Scientific).

For growth experiments, pre-cultures grown for 12 hours were diluted to a starting OD₆₀₀ of 0.01 with fresh medium containing different concentrations of the inducer theophylline (0-10 mM). Because theophylline is dissolved in DMSO, appropriate quantities of DMSO were added to all the cultures to account for any effect DMSO might have on growth.

### RNA Extraction and Quantitative Reverse Transcription PCR Analysis (RT-qPCR).

Prior to RNA extraction, 2 mL of C. sporogenes cultures grown in TYG to early stationary phase (OD₆₀₀≈2.5) were mixed with 4 mL of RNA Protect Bacteria reagent (Qiagen). Total RNA was extracted using the FastRNA Pro Blue kit (MP biomedicals), according to the manufacturer's instructions. Purified RNA was DNase-treated using the RQ1 RNase-Free DNase kit (Promega). cDNA synthesis was performed on 1 μg of RNA using the Omniscript RT kit (Qiagen). 5 μL of 1:10 diluted cDNA mixtures were used to perform qRT-PCR analysis using the Power SYBR Green Master Mix (Thermo Fisher Scientific) on a Light Cycler 480 II (Roche). cDNA synthesis reactions containing no reverse transcriptase were included as a control for genomic DNA contamination. Primer efficiencies were calculated for each primer set prior to use. qRT-PCR was performed on cDNA isolated from three biological replicates and in technical duplicates for each cDNA sample and primer pair. Results were calculated according to the E-method⁵⁸ and normalized to the 16Srrn and gyrA genes.

### HPLC/Ms Analysis.

To analyse if C. sporogenes consumes theophylline, 12 hour cultures were diluted to a staring OD₆₀₀ of 0.01. At an OD₆₀₀ of ≈0.5 cultures were induced with 2 mM theophylline. 1 mL samples were taken immediately and then at 2, 4, 6, 8, 10 and 20 hours after supplementation of theophylline. Samples were centrifuged for 10 min and 10000×g. Supernatants were used to determine the concentration of theophylline using an Alliance HT 2795 HPLC (Waters corporation) coupled to a Micromass Quattro LC Mass Spectrometer (Waters Wilmslow) in positive mode from m/z 70 to 500 for all samples at a scan rate of 1 cycle/s and equipped with an electrospray source. The general parameters were as follows: capillary voltage 2000 V, sampling cone 30 V, source temperature 130° C., desolvation temperature 350° C., cone gas flow 64 L/h and desolvation gas flow 621 L/h.

Prior to ionization, chromatographic separation was achieved using a Supelco Ascentis Express HPLC column (100 mm×3 mm, 2.7 μM, Sigma Aldrich). The mobile phase consisted of (A) water with 0.1% v/v formic acid and (B) methanol with 0.1% v/v formic acid. In all HPLC runs the elution gradient started at 95% A, 5% B increasing to 10% A, 90% B, followed by a 5 min re-equilibration period. A sample volume of 10 μL was injected for each HPLC run. The column was operated at 40° C. with a flow rate of 0.4 mL/min. The HPLC run contained blanks and the sample-relevant standard solution. Samples and standards were filtered using a 0.2 μM filter.

### Conjugation/Transformation Efficiency Determination and Mutant Screening.

For conjugation efficiency assays, the donor strain, E. coli CA434 (in triplicates), was grown overnight at 30° C. in LB supplemented with kanamycin and chloramphenicol. For each replica, two 1 mL cultures were centrifuged at 6,000×g for 1 min and then washed once with Phosphate-Buffered Saline (PBS). After a second centrifugation step, one of the two cultures was used to quantify the donor by plating the appropriate serial dilutions onto LB plates. The second culture was transferred to the anaerobic cabinet and mixed with 200 μL of a 12-hour C. sporogenes culture. The mixture was spotted onto the TYG medium supplemented with glucose. After 24 hours, cells were harvested, re-suspended in 500 μL of PBS and plated onto media supplemented with thiamphenicol and D-cycloserine, in the presence and absence of the inducer theophylline. After 24-48 hours of incubation at 37° C., colonies were counted and conjugation efficiency was calculated as total transconjugants per 1 mL of donor strain.

Transformation efficiency in C. pasteurianum was determined as the average of three independent transformations with 4 μg of plasmid DNA.

Single colonies were picked randomly and screened for desired mutants using colony PCR and specific flanking primers. In all cases, mutations were further confirmed with Sanger sequencing (data not shown).

5′-Terminal mRNA Analysis

To determine the transcriptional start site (TSS) of P_{fdx}, we sequenced the 5′-terminal end of the untranslated region (5′UTR) of the mRNA. Total RNA was extracted using the High Pure Isolation Kit (Roche). poly-T-cDNA was obtained using the 5′RACE kit 2^{nd} Generation (Roche) and the Expand High Fidelity PCR System (Roche) as per manufacturer's instructions. The kit requires three specific primers (IC263-3, IC264-r and IC265-r), all of them complementary to the mRNA transcript (catP). After amplification, the TSS was determined according to 5′UTR and the putative −10 and −35 boxes on the basis of typical characteristics of bacterial promoters.

### Tables

TABLE 1
Nucleotide sequences of riboswitches that were used in this study. The
aptamer sequence is underlined. The translational start is bold and italicised.
Modifications of the parent riboswitch E* are indicated in red. Rational
modifications were done towards known strong RBS sequences in the genus Clostridium.

| Riboswitch | Sequence (5′→3′) | Modification |
|---|---|---|
| D | GGUGAUACCAGCAUCGUCUUGAUGCCCUUGGCAGCACCCUGCUAAGGUAACAACAAG [symbol] | Riboswitches |
| E | GGUGAUACCAGCAUCGUCUUGAUGCCCUUGGCAGCACCCUGCUAAGGAGGUAACAACAAG | developed |
| | [symbol] | by Gallivan |
| E* | GGUGAUACCAGCAUCGUCUUGAUGCCCUUGGCAGCACCCUGCUAAGGAGGCAACAAC [symbol] | and coworkers⁵ |
| F | GGUGAUACCAGCAUCGUCUUGAUGCCCUUGGCAGCACCCUGCUAAGGAGGUAACAAC [symbol] | This study |
| G | GGUGAUACCAGCAUCGUCUUGAUGCCCUUGGCAGCACCCUGCUAAGGAGGUAACUUA [symbol] | |
| H | GGUGAUACCAGCAUCGUCUUGAUGCCCUUGGCAGCACCCUGCUAAGGAGGUGUGUUA [symbol] | |
| I | GGUGAUACCAGCAUCGUCUUGAUGCCCUUGGCAGCACCCUGCUAAGGAGGUCAACAAG [symbol] | |

TABLE 2
List of strains used in this study

| | | Reference |
|---|---|---|
| Strain | Relevant characteristics | source |
| E. coli TOP10 | F-mcrA Δ(mrr-hsdRMS-mcrBC) ϕ80lacZΔM15 ΔlacX74 | Invitrogen |
| | recA1 deoR araD139 Δ(ara-leu)7697 galU galK rpsL (StrR) | |
| | endA1 nupG | |
| E. coli CA434 | hsd20(rB-, mB-) recA13 rpsL20 leu proA2 with IncPb | 6 |
| | conjugative plasmid R702 | |
| E. coli TOP10-pCR1 | strain harbouring plasmid CR1 with M. Bepl methylase | 7 |
| Clostridium sporogenes | WT strain | ATCC |
| NCIMB 10696 | | |
| C. spo-ΔspollR | spollR deletion | This study |
| C. spo-ΔspollE | spollE deletion | This study |
| C. spo-spollE::ARC | spollE truncation and insertion of the ARC | This study |
| C. spo-Δc16380 | c16380 deletion | This study |
| C. spo-*c03700 | silent mutation in c03700 | This study |
| C. spo-Δc01750 | c01750 deletion | This study |
| C. spo-Δc04580::cargo | c04580 deletion and insertion of 1 kbp cargo | This study |
| C. spo-*c14540::cargo | silent mutation in c14540 and insertion of 0.8 kbp cargo | This study |
| C. spo-Δc05250- | deletion of c05250-c05270 operon and insertion of 1 kbp | This study |
| Δc05270 | cargo | |
| C. spo-Δc14780 | c14780 deletion | This study |
| C. spo-pyrE::cargo | pyrE deletion and insertion of 0.8 kb cargo | This study |
| C. spo-c31080 | c31080 deletion | This study |
| C. spo-c29800 | c29800 deletion | This study |
| Clostridium pasteurianum | hypertransfornnable strain based on DSM 525 | 7 |
| DSM 525-H1 | | |
| C. past-ΔspollE | spollE deletion | This study |
| Clostridium difficile 630 | WT strain | ATCC |
| C. diff-ΔspollE | spollE deletion | This study |
| Clostridium botulinum | WT strain | ATCC |
| ATCC 3502 | | |
| C. bot-ΔspollE | spollE deletion | This study |

TABLE 3
Media composition for Clostridium growth

| Name | Composition (g/L) | | Organism |
|---|---|---|---|
| TYG | Tryptone | 30 | C. sporogenes and C. |
| | Yeast extract | 20 | botulinum grown in solid or |
| | Na-thioglycolate | 1 | liquid media |
| | (if solid) Agar | 10 | |
| 2xYPG | Peptone | 16 | C. posteurianum grown in |
| | Yeast extract | 10 | liquid medium |
| | NaCl | 5 | |
| | Glucose | 25 | |
| | pH 6.2 | | |
| RCM agar | Yeast extract | 3 | C. posteurianum grown in |
| | Beef extract | 10 | solid medium |
| | Peptone | 10 | |
| | Glucose | 5 | |
| | Soluble starch | 1 | |
| | NaCl | 5 | |
| | Na-acetate | 3 | |
| | Cysteine | 0.5 | |
| | hydrochloride | | |
| | Agar | 15 | |
| | pH 6.8 | | |
| BHIS | Brain infusion solids | 12.5 | C. difficile grown in solid or |
| | Beef heart infusion | 5 | liquid media |
| | solids | | |
| | Protease peptone | 10 | |
| | Glucose | 2 | |
| | NaCl | 5 | |
| | Na₂HPO₄ | 2.5 | |
| | (if solid) Agar | 15 | |

TABLE 4
List of primers used in this study

| | | |
|---|---|---|
| | | Additional |
| Primer | Secuence (5′→3′) | information |

Riboswitch

| | |
|---|---|
| IC101-f | ATATGCGGCCGCGTGTAGTAGCCTGTGAAATAAGTAA |
| IC102-r | ATATGGTGTCACCATTAACACACCTCCTTAAAAATTACAC |
| IC103-f | TATAGGTCTCAATGGTATTTGAAAAAAT |
| IC104-r | TATACTCGAGTTAACTATTTATCAATTCCTG |
| IC105-f | CAGCGGCCGCATGGTATTTGAAAAAATTGATAAAAA |
| IC106-r | GGCGGTACCGATTTATATTTTACCATGTTTTTATTAATTT |
| IC107-f | CCCGGTACCAATACGACTCACTATAGGTTCCGGTGATACCAGCATCGTCTTGATGCCCTTG |
| IC108-r | CCGGTCTCACCATGTTGTTGCCTCCTTAGCAGGGTGCTGCCAAGGGCATCAAGACGATGCT |
| IC109-r | CCGGTCTCACCATGTTGTTGCCTCCTTAGCAGGGTGCTGCCAAGGGCATCAAGACGATGCT |
| IC110-r | CCGGTCTCACCATCTTGTTGTTACCTCCTTAGCAGGGTGCTGCCAAGGGCATCAAGACGATGC |
| IC111-r | CCGGTCTCACCATGTTGTTACCTCCTTAGCAGGGTGCTGCCAAGGGCATCAAGACGATG |
| IC112-r | CCGGTCTCACCATTAAGTTACCTCCTTAGCAGGGTGCTGCCAAGGGCATCAAGACGATGC |
| IC113-r | CCGGTCTCACCATTAACACACCTCCTTAGCAGGGTGCTGCCAAGGGCATCAAGACGATGC |
| IC114-r | CCGGTCTCACCATCTTGTTGACCTCCTTAGCAGGGTGCTGCCAAGGGCATCAAGACGATGC |
| IC115-r | CCGGTCTCACCATTGGTCGTACACTCCCTTAGCAGGGTGCTGCCAAGGGCATCAAGACGATGC |
| IC116-r | CCCGGTCTCTATTGAAAAATTACACAACTTTATACGATTTAT |
| IC117-r | GGTCTCACAATACGACTCACTATAGGTGATACCAGCATCGTCTTGATGCCCTTG |
| IC118-r | AAACTTAACTTCATGTTAAAAACTTGTTAAAATATAAATCGTATAAAGTTGTGTAATTT |
| IC119-r | ATTTATATTTTAACAAGTTTTTAACATGAAGTTAAGTTTTTTTTATATAACTGTATAACC |

RiboCas

| | | |
|---|---|---|
| IC201-f | ATATATATGCGGCCGCTGTATCCATATGACCATGATTACGAATTCTCAGTCACCTCCTAGCTGAC | |
| | T | |
| IC202-r | GCTATGGTCTCCATGGATAAGAAATACTCAATAGGCTTAG | |
| IC203-f | GGCCTCTAGAGTGTAGTAGCCTGTGAAATAAGTAAGGAAAAAAAAGAAGTAAGTGTTATATAT | |
| | GATG | |
| IC204-f | GGCCGGCGCGCCGTGTAGTAGCCTGTGAAATAAGTAAGGAAAAAAAAGAAGTAAGTGTT | |
| IC205-f | ATATATTCTAGATTTATATTTAGTCCCTTGCC | |
| IC206-r | GGCCGGCGCGCCATTGACGTCTATGTCGACGAAAACTCCTCCTTAAGA | |
| IC207-f | GTATCATCTAGAGTAAAACGACGGCCAGTTTGACAGCTAGCTCAGTCCTAGGTATAATACTAGT | |
| IC208-r | GTATCAGGCGCGCCATTGACGTCTATACTAGTATTATACCTAGGACTGAGCTAGCTGTCAAACT | |
| | GG | |
| IC209-r | GGCCCCGGGACGTCATAAAAATAAGAAGCCTGCAAATGCAGGCTTCTTATTTTTATAAAAAAAG | sgRNA-r |
| | CACCGACTCGGTGCCACTTTTTCAAGTTG | |

C. sporogenes-spoIIR deletion

| | | |
|---|---|---|
| IC211-f | GGCCGACGTCTCATTAGATGCATATTCAATGCAGGATAGTATAG | LHA^{a} |
| IC212-r | ATTTTCAATACAGAGGTTGATCTTATTTATTAGTTATTATTACCAAATTTTATAGTTATA | |
| IC213-f | ATAAATAAGATCAACCTCTGTATTGAAAATAATACAGAGTTTTTTTATAATAAAA | RHA^{b} |
| IC214-r | AAGGCGGCGCGCCATTATGAACTACAAACTTTCTCATTTAATAGATGAATTTGACC | |
| IC215-f | CCGGGTCGACGGTAATATAACATTGCCACAGTTTTAGAGCTAGAAATAGCAAGTTAAAATAAGG | sgRNA-f-SalI |
| | CTAGTCCGTTATCAACTTGAAAAAGTGGCACCGAGTCGGTGCTTTTTTT | |
| IC216-f | GGCCACTAGTGGTAATATAACATTGCCACAGTTTTAGAGCTAGAAATAGCAAGTTAAAATAAGG | sgRNA-f-SpeI |
| | CTAGTCCGTTATCAACTTGAAAAAGTGGCACCGAGTCGGTGCTTTTTTT | |
| IC247-f | TTTCCGTTCTAGGAGTGGATGCGGGAAAGGA | Screening |
| IC248-r | TGACCATTCAGAGGATGATGAAGGTTTAGCC | primers |

C. sporogenes-spoIIE deletion

| | | |
|---|---|---|
| IC217-f | GGCCGGCGCGCCGCTGGAGTGGCGGAACTGGCAGACGCACAGGACTTAAAATCCTGCGGGCC | LHA |
| | TAACAG | |
| IC218-r | TATATTCTCTTTCTATATTTTAATATAATTGCATAATAATTAATCACCCCCATTAAGTTACTTCTCA | |
| | ATTATATCA | |
| IC219-f | ACTTAATGGGGGTGATTAATTATTATGCAATTATATTAAAATATAGAAAGAGAATATAAAGTTA | RHA |
| | AGTAGTAAATACTTA | |
| IC220-r | GGCCGACGTCTCATCATCAATATTTTTCACAATAATTTTCTATATCTTCTCTTAATACATTTATTAA | |
| | AGGTCTTATATATACA | |
| IC221-f | GGCCGTCGACTTAATTATAGGATATGTAAAGTTTTAGAGCTAGAAATAGCAAGTTAAAATAAGG | sgRNA-f |
| | CTAGTCCGTTATCAACTTGAAAAAGTGGCACCGAGTCGGTGCTTTTTTT | |
| IC249-f | ATATAATTATTTATGTCGTCAGCAATGAGAC | Screening |
| IC250-r | CTCTCTTATCTATTATTACCCTTTGTGTT | primers |

C. sporogenes-ARC integration

| | | |
|---|---|---|
| IC222-f | GGCCGACGTCATGCAATATGGCGCAGAACTTTTACCATACCA | LHA |
| IC223-r | GGCCGCTCTTCTAAGTCATCATCATATCATAGTAATAGATATTAAATTCATTAAATTT | |
| IC224-f | GGCCGCTCTTCCCTTTAAGGAGGTGTGTTACATATGAACAAAAATATAAAATATTCTCAAAACTT | ermR |
| | TTTAACGAGTGAAAAAGTACTCAACCAAATA | |
| IC225-r | GTACCGAGCTCGAATTCGTAATTTATTATTATTTCCTCCCGTTAAATAATAGATAACTATTAAAAA | |
| | TAGACAATACTTG | |
| IC226-f | TCTATTATTTAACGGGAGGAAATAATAATAAATTACGAATTCGAGCTCGGTACCCGGGGATCCT | lacZα MCS |
| | CTAGAGTCGACGTCACGCGTCC | |
| IC227-r | AACACACCTCCTTAAATCACTGCCCGCTTTCCAGTCGGGAAACCCTAGCGCCATTCGCCATTCAG | |
| | GCTGCGCAACTGTTGGGAAGGGCGATCGGTGCGGGCCTCTTCGC | |
| IC228-f | GGTTTCCCGACTGGAAAGCGGGCAGTGATTTAAGGAGGTGTGTTACATATGTTACGTCCTGTAG | gusA |
| | AAACCCCAACCCGTGAAATCAAAAAACTCG | |
| IC229-r | GCTCTTCTCATTGTTTGCCTCCCTGCTGCGGTTTTTCACCGAAGTTCATGC | |
| IC230-f | GGCCGCTCTTCCATGAGGGAAGTACTAGTGCTTCAGCTATCGGTGTGGCT | RHA |
| IC231-r | GGCCGGCGCGCCTTTCATTTATTATATAATTATTTACTATATCTTC | |

C. pasteurianum-spoIIE deletion

| | | |
|---|---|---|
| IC232-f | GGCCGGCGCGCCGCCGAAGTGGCGGAACTGGCAGACGCACAGGACTTAAAATCCTGCGGTGCT | LHA |
| | TAAACCACCGTAC | |
| IC233-r | ATTCAATTAATAAACAACATTTTAATACAATTGCATACAAACATCTCCCAACCTATAAACTATTTT | |
| | TATTATAA | |
| IC234-f | GTTTATAGGTTGGGAGATGTTTGTATGCAATTGTATTAAAATGTTGTTTATTAATTGAATGAGAA | RHA |
| | TTTATAA | |
| IC235-r | GGCCGACGTCTCATCATCATGGATTAATGTGTTCCTTCTTACAATAGCTTTCTATTTCTTCTCTTCC | |
| | TATTTTTATT | |
| IC236-f | GGCCGTCGACGGAAACTCCTAAATATCACGGTTTTAGAGCTAGAAATAGCAAGTTAAAATAAG | sgRNA-f |
| | GCTAGTCCGTTATCAACTTGAAAAAGTGGCACCGAGTCGGTGCTTTTTTT | |
| IC253-f | ATGATATATAATAGTTTCTGTTGCAGTAAG | Screening |
| IC254-r | GTTATCTACATTTACTGTATCAGATAATC | primers |

C. difficile-spoIIE deletion

| | | |
|---|---|---|
| IC237-f | GGCCGGCGCGCCGTTACGGGAATAGAAGAATCAAATTTGGGCTTTAAGGGTGTTTTAGAAGGA | LHA |
| | ATACAAGGTGATG | |
| IC238-r | ATACACATAATCTTGTAAACCTTATTTTGTTTGCATAAAAAAACCTCCTCTTTTGTTTATATTAAG | |
| | AATTGTAAC | |
| IC239-f | ATAAACAAAAGAGGAGGTTTTTTTATGCAAACAAAATAAGGTTTACAAGATTATGTGTATAAGA | RHA |
| | TGTGGATAACTTG | |
| IC240-r | GGCCGACGTCTCACCTCATCAATCAAGTTTTTCTTTAATGTTTAAGGCAAAAGACAAATGTGTTG | |
| | GCGATTTTGTTA | |
| IC241-f | GGCCGTCGACGGAATTTTAGATTTAAAACGGTTTTAGAGCTAGAAATAGCAAGTTAAAATAAGG | sgRNA-f |
| | CTAGTCCGTTATCAACTTGAAAAAGTGGCACCGAGTCGGTGCTTTTTTT | |
| IC251-f | TTCTGATAAGTGTAATGATGAAAAGTGTG | Screening |
| IC252-r | CTAACTCTAAATCTGAACAAATCCCCATA | primers |

C. botulinum-spoIIE deletion

| | | |
|---|---|---|
| IC242-f | GTTCATGGCGCGCCCTTCTAAATAGTTATTGTCTTTAGAAACTATATTAG | LHA |
| IC243-r | GCTATGGTCTCCTTATACTAAAATATAGAAAGAGAATATAAAGTTAGGTAG | |
| IC244-f | GCTACTGGTCTCAATAATTGCATAATAATTAATCACCCCCATTAAG | RHA |
| IC245-r | GTAGCCGACGTCCAGCGATGAGACATAAACATAAAAC | |
| IC246-f | TTTTCGTCGACTTTTATAAAGCGAGGAACTGGTTTTAGAGCTAGAAATAGCAAGTTAAAATAAG | sgRNA |
| | GCTAGTCCGTTATCAACTTGAAAAAGTGGCACCGAGTCGGTGC | |
| IC255-f | GAAATAAAGTGTTGACATAGTAAAGTTTG | Screening |
| IC255-r | AAAGCTCTCTTATCTATTATTACCCTTTG | primers |

qPCR

| | | |
|---|---|---|
| IC257-f | CTACTTTGCAAGTGTACCTTG | Target gene |
| IC258-r | ATCCCCAATTCACCATCTTG | |
| IC259-f | AGCGGTGAAATGCGTAGAGA | 16Srrn |
| IC260-r | GGCACAGGGGGAGTTGATAC | |
| IC261-f | GAAAGGGTATTCAAGCCACATC | gyrA |
| IC262-r | CTATCGGTGTGGCTATGGGGGCT | |

5′ RACE

| | |
|---|---|
| IC263-r | AAATGTGAAATCCGTCACATACT |
| IC264-r | CAAAGGAAGTATAATTTTGTTATCTTC |
| IC265-r | CTGCTAGCTTTCTGCAAATTCAGATTAAA |

^{a}LHA, left homology arm
^{b}RHA, right homology arm

TABLE 5
List of plasmids used in this study

| | | Reference |
|---|---|---|
| Plasmid | Relevant characteristics | source |
| pMTL8225 | Erm^{R}; modular vector for the evaluation of inducible systems | 1 |
| pMTL-IC101 | Erm^{R}; P_{fdx}-catP | this study |
| pMTL-IC201 | Erm^{R}; Ph4-catP | this study |
| pMTL-IC001 | Erm^{R}; promoterless pMTL-1C101 | this study |
| pMTL-IC111-D | Erm^{R}; P_{fdx}-RbD-catP | this study |
| pMTL-IC111-E | Erm^{R}; P_{fdx}-RbE-catP | this study |
| pMTL-IC111-E* | Erm^{R}; P_{fdx}-RbE*-catP | this study |
| pMTL-IC111-F | Erm^{R}; P_{fdx}-RbF-catP | this study |
| pMTL-IC111-G | Erm^{R}; P_{fdx}-RbG-catP | this study |
| pMTL-IC111-H | Erm^{R}; P_{fdx}-RbH-catP | this study |
| pMTL-IC111-I | Erm^{R}; P_{fdx}-RbI-catP | this study |
| pMTL-IC111-J | Erm^{R}; P_{fdx}-RbJ-catP | this study |
| pMTL-IC201-E | Erm^{R}; Ph4-RbE-catP | this study |
| pMTL-IC201-G | Erm^{R}; Ph4-RbG-catP | this study |
| pMTL-IC201-F | Erm^{R}; Ph4-RbF-catP | this study |
| pMTL-IC121-E | Erm^{R}; P_{fdx}*-RbE-catP | this study |
| pMTL-IC121-G | Erm^{R}; P_{fdx}*-RbG-catP | this study |
| pMTL-IC121-H | Erm^{R}; P_{fdx}*-RbH-catP | this study |
| pMTL-IC221-E | Erm^{R}; Ph4*RbE-catP | this study |
| pMTL-IC221-G | Erm^{R}; Ph4*-RbG-catP | this study |
| pMTL-IC221-F | Erm^{R}; Ph4*-RbF-catP | this study |
| pMTL8315 | CM^{R}; modular vector used as a chassis for the RiboCas system | 1 |
| pRECasC | CM^{R}; P_{fdx}*-RbE | this study |
| pRECasG-llR | CM^{R}; P_{fdx}*-RbE-P_{araE}-SgRNA (spollR) | this study |
| pRECas1-llR | CM^{R}; P_{fdx}*-RbE-P_{araE}-SgRNA-editing template (spollR) | this study |
| pRECas2-llR | Cm^{R}; P_{fdx}*-RbE-J23119-sgRNA-editing template (spollR) | this study |
| pRGCasC | CM^{R}; P_{fdx}*-RbG | this study |
| pRGCasG-llR | CM^{R}; P_{fdx}*-RbG-P_{araE}-SgRNA (spollR) | this study |
| pRGCas1-llR | CM^{R}; P_{fdx}*-RbG-P_{araE}-SgRNA-editing template (spollR) | this study |
| pRGCas2-llR | Cm^{R}; P_{fdx}*-RbG-J23119-sgRNA-editing template (spollR) | this study |
| pRECasG-llE | Cm^{R}; P_{fdx}*-RbE-P_{araE}-SgRNA (spollE) | this study |
| pRECas1-llE | Cm^{R}; P_{fdx}*-RbE-P_{araE}-SgRNA-editing template (spollE) | this study |
| pRECas1-llEin | Cm^{R}; P_{fdx}*-RbE-P_{araE}-SgRNA (spollE)-editing template (ARC) | this study |
| pRECasG-llE-Cpas | Cm^{R}; P_{fdx}*-RbE-P_{araE}-SgRNA (spollE)- C. posteurianum | this study |
| pRECas1-llE-Cpas | Cm^{R}; P_{fdx}*-RbE-P_{araE}-SgRNA-editing template (spollE)-C. | this study |
| | posteurianum | |
| pRECasG-llE-Cdif | Cm^{R}; P_{fdx}*-RbE-P_{araE}-SgRNA (spollE)- C. difficile | this study |
| pRECas1-llE-Cdif | Cm^{R}; P_{fdx}*-RbE-P_{araE}-SgRNA-editing template (spollE)-C. | this study |
| | difficile | |
| pRECasG-llE-Cbot | Cm^{R}; P_{fdx}*-RbE-P_{araE}-SgRNA (spollE)-C. botulinum | this study |
| pRECas1-llE-Cbot | Cm^{R}; P_{fdx}*-RbE-P_{araE}-SgRNA-editing template (spollE)-C. | this study |
| | botulinum | |

TABLE 6
Summary of genome editing results in different Clostridium species

| | | Modification | | | |
|---|---|---|---|---|---|
| | | Deletion | Insertion | Protospacer | |
| Strain | Target | (kbp) | (kbp) | sequence | ^{a}efficiency (%) |
| C.posteruianum DMS 525 | spoIIE | 2.4 | — | ggaaactcctaaatatcacg | 100 |
| C.difficile 630 | spoIIE | 2.4 | — | ggaattttagatttaaaacg | 100 |
| C.botulinum ATCC 3502 | spoIIE | 2.4 | — | ttttataaagcgaggaactg | 100 |
| C.sporogenes NCBI 10696 | spoIIE | 2.4 | — | ttaattataggatatgtaaa | 100 |
| | spoIIE | — | 2.9 | ttaattataggatatgtaaa | 100 |
| | spoIIR | 0.7 | — | ggtaatataacattgccaca | 100 |
| | c16380 | 0.63 | — | acagttttttcaattttcgg | 100 |
| | c03700 | silent | silent | ttacaactactatccaaaga | 94 |
| | | mutation | mutation | | |
| | c01750 | 1.4 | — | aagtactccgattaaaacag | 84 |
| | c04580 | 1 | 1 | ggtttagaagaaatagcggg | 83 |
| | c14540 | — | 0.8 | gacaaaacaagtcaaataga | 67 |
| | c05250 | 8.5 | 1 | ggactcttaaccccaactaa | 57 |
| | c14780 | 0.6 | — | ccaaatatagtccaacctaa | 56 |
| | pyrE | 0.4 | 0.8 | atagtaggaccagctatggg | 42 |
| | c31080 | 0.24 | — | gttgttttaattgatcacaa | 38 |
| | c29800 | 1.3 | — | acttgtagcggcagaagcac | 36 |

^{a}(Number of corretly edited transformants/total number of transformants screened) × 100

### EXAMPLES

Demonstration that Riboswitches Work in Clostridium

Diverse theophylline-dependent riboswitches work in both Gram-positive and Gram-negative bacteria (Topp, S. et al. Appl. Environ. Microbiol. 76, 7881-4 (2010)), however the data presented here is the first to show the performance of this inducible translational regulatory system in Clostridium sporogenes NCIMB 10696. Three known riboswitch sequences were selected, riboswitch-D, -E and -E* (sequence ID Nos: 1, 2 and 3 respectively), which were expected to exhibit higher translation efficiencies in the ‘on’ state. In previous publications, riboswitch E* was shown to be the best theophylline-dependant riboregulator in Gram-positive bacteria, thanks to its high dynamic range and its very low basal expression. 4 new riboswitches (named -F to -I, sequence ID Nos: 4 to 7) were constructed by rationally modifying the space between the Shine Dalgarno sequence (SD) and the translational start site of the original riboswitch E* (Table 1). A reporter plasmid, pMTL-IC101, derived from the pMTL82251 vector (Heap et al, J. Microbiol. Methods 78, 79-85 (2009)) was used as a chassis for all the constructs created thereof (FIG. 1a). This backbone contains the native ferredoxin promoter (P_{fdx}, associated with the protein coding gene Clspo_c0087) upstream of catP, which encodes the reporter Chloramphenicol Acetyl Transferase (CAT, EC: 2.3.1.28) and serves as a reference to compare CAT expression measurements. As a default, the riboswitches, preceded by a linker sequence, were genetically fused to the core region (−35 and −10) of the strong P_{fdx}, just downstream of the transcription start site (TSS) and excluding the native 5′UTR sequence. Constructs, named pMTL-IC111-D to -J, were conjugated into C. sporogenes and induced at early-exponential phase (OD₆₀₀≈0.5) with 2 mM theophylline. CAT activity was determined on cell lysates from stationary cultures in the presence and absence of the inducer. As a control, a promoterless backbone, pMTL-IC001, was assembled to detect background reporter gene expression. A detailed list of plasmids used in this study is provided in Table 5.

The synthetic theophylline responsive riboswitch is composed of an aptamer and a synthetic SD (FIG. 1b). Theoretically, transcription of the riboswitch under the control of P_{fdx} occurs in a constitutive manner during cell growth. However, the synthetic SD sequence located downstream of the aptamer is sequestered via pairing with the stem of the riboswitch, resulting in translational block. The binding of theophylline releases the SD by altering the downstream base pairing. As a consequence, gene translation occurs when the ribosome binds to the SD. As shown in FIG. 2a, strains harbouring riboswitches E, E*, F, G, H and I exhibited statistically significant induction in C. sporogenes after addition of 2 mM theophylline to the culture. In particular, riboswitch G outperformed previous theophylline-dependant riboswitches, demonstrating higher levels of CAT activity, low leakage expression and the strongest activation ratio (FIG. 2b). In all cases, the incorporation of any riboswitch in the 5′UTR led to a strong reduction in CAT activity; this agrees with previously published studies, indicating that secondary structures near the RBS play a major role in the translation of the downstream mRNA. CAT activity of cells harbouring the control pMTL-IC001 was similar to that from cultures lacking the reporter backbone (wildtype, WT), indicating no detectable transcriptional read-through.

### Riboswitches can be Adjusted to the Desired Regulatory Window

Due to the importance of having inducible systems where gene expression can be controlled within a large regulatory window, the expression pattern of riboswitches E, G and H when placed downstream of the core region of a synthetically weakened Plea, named Ph4 was compared. Ph4 was generated by replacing the core elements −10/−35 of P_{fdx} with the same regulatory elements of the constitutive ptb promoter from C. acetobutylicum ATCC 824 (P_{ptb}, associated with the protein coding gene Ca_3076) (FIG. 3a). Also, to further examine the regulatory response of the theophylline responsive riboregulators in Clostridium, riboswitches E, G and H were fused to the promoters P_{fdx} and Ph4 retaining the bases downstream the TSS but excluding their native SD sequences. These sequences, named P_{fdx}* and Ph4*, maintain the full upstream region, including the core region (−35 and −10), the TSS and the space between the TSS and the native SD. Constructs were designed to express the reporter gene catP, conjugated into C. sporogenes and induced with 2 mM theophylline. As shown in FIG. 3b, the combination of different riboswitches with various promoters and two different 5′UTRs allows the expansion of the regulatory range, providing a library of theophylline-inducible switches suitable for applications where protein yield is crucial as well as for the expression of detrimental or toxic proteins. The combination of any riboswitch with the weak promoter Ph4 led to the lowest expression levels. This was also the case when the native 5′UTR sequence was retained, establishing a direct correlation between detected CAT activity and promoter strength (P_{fdx}>Ph4). However, basal expression appears to be compromised when high expression levels are achieved, resulting in increased leakiness in those constructs that exhibited higher expression levels (i.e. with P_{fdx}*-G). Expression of catP was always higher when the native sequence downstream of the TSS was maintained. Since riboswitch-G showed the highest induction level of the analysed riboregulators, it was subjected to further characterization.

### Functional Characterization of the Theophylline Responsive Riboswitch in Clostridium

To assess the dose dependency of riboswitch regulation, strains harbouring the riboswitch G downstream of P_{fdx} or P_{fdx}* were cultivated with increasing concentrations of theophylline, ranging from 0.1 to 10 mM (FIG. 5a). Utilization of increasing concentrations of theophylline entailed higher levels of gene expression; however, concentrations higher than 5 mM resulted in a reduction of growth, in agreement with previously reported results in other Gram-positive and -negative bacteria (FIG. 5b). Also, the translational switches were analysed for CAT expression over time in the absence and presence of 2 mM theophylline. As shown in FIG. 5c, CAT expression above the level of the uninduced culture was already detectable 30 min after induction (4.5 hours of growth). The maximum level of reporter gene expression was reached at 8 and 10 hours when using P_{fdx} and P_{fdx}* respectively; although both have a similar profile of induction, after 24 hours, CAT expression was reduced by 75% in P_{fdx} whereas only by 40% in P_{fdx}*. Given that in both cases the translated product is the same (CAT), the results suggest that the reversal of induction is strongly dependent on the abundance or stability of the mRNA.

As requisite of a good inducer, it was ascertained that theophylline is not metabolized by Clostridium sporogenes. Clostridial cells were cultivated in TYG medium, with theophylline added to the culture to a final concentration of 2 mM. The concentration of theophylline was monitored by HPLC/MS (high-performance liquid chromatography-mass spectrometry) analysis of cell-free supernatant samples over time. Results showed that the concentration of theophylline remains constant over the course of the experiment, with a slight increase of theophylline in the supernatant culture 20 hours after induction, possibly due to the release of the inducer from the cells after lysis (FIG. 5d).

### Application of Riboswitches to Create a Highly Efficient CRISPR/Cas9-Based Tool for Clostridium

The data below demonstrates that using the above described tightly inducible gene expression system in Clostridium an efficient Clostridial editing CRISPR tool can be provided which circumvents the obstacles previously seen for several reasons. Firstly, the theophylline responsive riboswitch can be designed to have the desired regulatory window, whereby there should be very low basal expression of Cas9 in the absence of inducer, allowing homologous recombination to occur before Cas9 mediates the site-specific DSB; after induction, an adequate level of gene expression occurs whilst minimizing the toxicity associated with Cas9. Secondly, riboswitches are smaller structures compared to systems that require a transcription factor; in a riboswitch-based system, 84 nucleotides are enough to tightly control the expression of Cas9, shrinking the size of the plasmid employed and enabling higher transformation efficiencies. Thirdly, because repression occurs at the level of translation, the riboswitch system allows the use of high-copy origins of replication, avoiding the undesired effects linked to read-through transcription on the plasmid backbone and facilitating the processes of cloning, screening and sequencing. Finally, and due to the aforementioned characteristics, a genetic construct according to the invention permits the confinement of all the essential components of a functional genome editing tool to the same plasmid. FIG. 6 illustrates the assembly of the RiboCas vector series a series of vectors containing a genetic construct according to the invention, generically named pRXCasN vectors, where X and N refer to the riboswitch and the promoter driving the sgRNA respectively, and the general RiboCas-based editing process.

In order to have an appropriate control over cas9 expression, both in the uninduced state and after induction, two riboswitches were used, riboswitches E and G, both were located downstream of the promoter P_{fdx} (FIG. 2a). Riboswitch E had shown the tightest repression in the non-induced state amongst all tested riboswitches; on the other hand, riboswitch G showed higher background levels but increased expression when the inducer was added. Two promoters, the Clostridium acetobutylicum constitutive araE promoter (P_{araE}, associated with the protein coding gene Ca_1339) and the synthetic promoter J23119, were chosen to bring about the expression of the sgRNA. Both had been previously used for CRISPR strategies with Clostridium species. In all cases, the editing template comprised two homology arms composed of the regions of the chromosome needed to replace the target region. As a proof-of-principle, it was decided to target the spoIIR gene (Clspo_c01510, encoding the stage II sporulation protein R) as this gene had been previously disrupted in C. sporogenes (data not shown). The DNA modules encoding either riboswitch E or G, cas9, the sgRNA downstream of either P_{araE} or J23113 and the DNA editing template were inserted into the pCB102-based modular vector pMTL83151¹⁷, yielding the following pRXCasN vector series: pRECas1-IIR, pRECas2-IIR, pRGCas1-IIR and pRGCas2-IIR (Table S1). The same vectors lacking the editing homology arms were assembled to determine the killing capacity of Cas9, named pRECas1G-IIR, pRECas2G-IIR, pRGCas1G-IIR and pRGCas2G-IIR. Two vectors, pRECasC and pRGCasC, lacking both the homology arms and sgRNA, were also created as a control for conjugation efficiency comparisons.

Transformation of the resulting vectors into C. sporogenes was performed via conjugation, plating the mated cultures into media supplemented with chloramphenicol in the absence and presence of 5 mM theophylline. As expected, the different constructs yielded different conjugation efficiencies (FIG. 7a). The combination of riboswitch E and the promoter P_{araE} upstream of the sgRNA (pRECas1) provided the highest conjugation efficiency (1.21×10⁻⁷ CFU/donor; CFU: colony forming units), comparable to the 1.46×10⁻⁷ CFU/donor obtained with pRECasC (non-targeting Cas9). Transformation of the control plasmids, pRECasC and pRGCasC, resulted in similar colony counts on plates with an without theophylline (FIG. 7b), indicating a very low off-target activity in the absence of the targeting module. In all cases conjugation efficiency was lower if riboswitch G regulated the expression of Cas9, which implies a higher background expression level in the absence of the inducer and agrees with previous CAT results. No colonies were observed on plates supplemented with theophylline if expressing the sgRNA, whether or not the editing template was included, indicating highly efficient Cas9-mediated killing of host cells (FIG. 7c). These results also suggest that it could be possible to select mutant cells just after transformation if the homologous recombination event had occurred during the recovery time.

Individual colonies harbouring the different CRISPR vectors and the DNA editing template were picked and transferred to selective plates in the presence of theophylline. In all cases, amplicons of ˜2.2 kbp instead of ˜2.9 kbp size were detected after screening, implying the expected ˜0.7 kbp complete deletion of spoIIR and an editing efficiency of 100% (FIG. 7d). Desired deletions were confirmed by sequencing the PCR products. Screening of uninduced colonies generated the amplicon of the WT strain (2.9 kbp); sequencing of these PCR products did not show insertions/deletions. These results suggest that the dynamic range of riboswitch E is appropriate for a Cas9-base editing system, providing both undetectable off-target activity and high selection capacity. The consistency of these results was confirmed by three independent experiments, verifying the robustness of the genetic construct and its suitability for more ambitious genome editing strategies.

### Application of RiboCas for the Deletion and Integration of Larger Fragments

To further illustrate the versatility of the RiboCas system/genetic construct of the invention, attempts were made to both delete and integrate larger fragments into the genome of C. sporogenes. The spoIIE gene (Clspo_c37040), which is ˜2.4 kbp was selected as the target locus. Two sets of homology arms were designed to either delete the target sequence or to integrate a 3 kbp cassette that confers resistance to the antibiotic erythromycin once inserted into the genome (FIG. 8a), making the integration easily detectable. The editing templates and the sgRNA sequences targeting spoIIE were cloned into pRECas1 (the most efficient RiboCas system in C. sporogenes in terms of conjugation efficiency) generating pRECas1-IIE (deletion vector) and pRECas1_IIEin (integration vector). Both vectors were successfully conjugated into C. sporogenes. After conjugation, induction of cas9 expression was performed by re-streaking individual colonies harbouring pRECas1-IIE onto plates supplemented with thiamphenicol and 5 mM theophylline and colonies harbouring pRECas1-IIEin onto plates containing thiamphenicol, theophylline and erythromycin. PCR was performed on uninduced cultures and on colonies that had grown for 24 hours in the presence of the inducer. 100% of the screened colonies were confirmed as positive mutants for spoIIE deletion and integration of the antibiotic resistance cassette (ARC) respectively (FIG. 8b). Generally, PCR products obtained from colonies grown in the absence of the inducer corresponded to unedited cells, demonstrating the tightness of RiboCas system in the absence of the inducer. Only a few colonies (<10%) were composed of mixtures of mutant and WT cells.

### RiboCas as a Universal Clostridium Editing Tool

To determine the applicability of the theophylline responsive riboswitches and the RiboCas editing tool in other clostridia species, studies were undertaken to delete the same gene, spoIIE, in three different species, including the solventogenic C. pasteurianum and the pathogens C. difficile and C. botulinum. Previous work had enabled the use of Cas9 for editing purposes in C. pasteurianum; however, the strategies employed suffer from very low transformation efficiencies that might compromise more ambitious applications. This is also the case in C. difficile; which despite some limited success, the performance of CRISPR still remains unsatisfactory. In particular, in many cases conjugation efficiency of the CRISPR plasmid is very low. This is likely due to the high toxicity associated with the Cas9 endonuclease activity.

Vectors pRECas1, containing the homology arms and the sgRNA to target the spoIIE gene in each species were introduced into the host organisms Clostridium pasteurianum DSM 525-H1, Clostridium difficile 630 and the group I Clostridium botulinum strain ATCC 3502. C. pasteurianum was transformed via electroporation whereas C. difficile and C. botulinum were transformed via conjugation. Simultaneously, the control plasmid pRECas1C (RiboCas vector lacking both homology arms and sgRNA), was also introduced into the different hosts to determine the leakiness of the system and its impact on the transformation/conjugation process. All transformations were plated on media supplemented with thiamphenicol, both in the absence and presence of theophylline. Transformation of the targeting RiboCas vector in C. pasteurianum was only 20% lower than that of the control vector (5000 CFU/μg DNA in pRECas1 vs 6500 CFU/μg DNA in pRECas1C) (FIG. 9). On average a conjugation efficiency of 75 CFU/μg DNA was obtained on plates containing theophylline, demonstrating that the theophylline responsive riboswitch is functional in C. pasteurianum. PCR screening of these colonies confirmed the mutant genotype of the edited cells without the need of further re-streaking; the unedited phenotype was maintained on plates lacking the inducer. Colonies from non-induced plates were streaked onto plates containing both thiamphenicol and theophylline; subsequently, colony PCR confirmed the identity of the mutant phenotype in 24 out of the 24 colonies screened (100% efficiency) (Table 1). A similar transformation pattern was found when conjugating the RiboCas vectors into C. difficile and C. botulinum (data not shown), validating the induction ability of riboswitches and the efficiency of the RiboCas tool in both organisms. Screening of C. difficile colonies after induction confirmed an efficiency of 100% while maintaining the WT phenotype on colonies not exposed to theophylline (Table 6).

A summary of all gene editing attempts carried out for the validation of the RiboCas system in Clostridium, including insertions, deletions and nucleotide substitutions are summarized in Table 1. It is worth mentioning that, despite pRECas1 being the most efficient in this study (in terms of conjugation efficiency), vectors including the promoter J23119 may be suitable for organisms where P_{araE} is expected to be natively repressed (i.e. in C. acetobutylicum).

### Use of a Riboswitch to Control Sigma Factor Expression

To exemplify the use of a riboswitch to control the expression of an alternative sigma factor, botR from C. botulinum ATCC 19397 was placed under the control of the Plea promoter from Clostridium sporogenes NCIMB 10696 and a theophylline-responsive riboswitch (rb G; SEQ ID No. 5). In the present embodiment, expression of BotR would activate its cognate promoter Pn/nh, which would in turn bring about catP expression, quantifiable spectrophotometrically via a Chloramphenicol Acetyltransferase (CAT) enzymatic assay. The P_{fdx}-rbG-botR-T1-Pₙₜₙₕ-catP construct (FIG. 1) was integrated in the pyrE locus of the chromosome of a C. botulinum ATCC 19397 strain in which the entire toxin A gene cluster had been deleted (C. botulinum ATCC 19397 ΔClA). Therefore, the strain bore no other botR gene copy except for the one supplied with this construct. Integration was carried out using Allele-Coupled Exchange (ACE) via pyrE repair, as described by Heap, J. T. et al Nucleic Acids Res, 2012. 40(8): p. e59-e59.

As can be seen in FIG. 11, in uninduced state there is very low background CAT activity, comparable to the negative control sample which does not contain catP. In the absence of inducer, expression of BotR is translationally blocked due to the presence of the riboswitch. In turn, the absence of BotR prevents transcriptional activation of Pₙₜₙₕ, resulting in negligible expression of the CAT reporter. Expression upon induction is approximately 7-fold higher than in the absence of theophylline, in agreement with the expected/desirable dynamic range between the OFF and ON state, in the presence of a riboswitch.

This serves to demonstrate that a riboswitch as described in the present invention can be successfully employed to tightly control the expression of a sigma factor.

### CONCLUSIONS

This study shows the first implementation of synthetic riboswitches in the genus Clostridium and demonstrates their application in highly efficient genome editing. New and known theophylline-dependent riboswitches are demonstrated to function in C. sporogenes, with a response that is dependent on inducer concentration. In particular, the novel riboswitch G outperformed previously published riboswitches, exhibiting a large dynamic range and very low basal expression. By replacing the promoter and modifying the 5′UTR sequence located upstream of the riboswitch, a library of inducible switches with a full set of dynamic ranges were generated, suitable for applications where high levels of gene expression are needed as well as for the expression of toxic proteins. In all cases, the riboswitch was able to respond to the inducer theophylline, ensuring its performance independent of the genetic context.

To validate the usefulness of these tight riboregulators, a novel CRISPR Cas9-based system—RiboCas—was developed and shown to work in several clostridial species, including the non-pathogen C. sporogenes, the solventogenic C. pasteurianum and the pathogens C. difficile and C. botulinum. This novel system, using a genetic construct according to the invention, overcomes the main obstacles associated with Cas9 editing tools, including the very low transformation efficiencies and the inability to select only edited cells as a result of excessive Cas9 toxicity. For this reason, temporal and minimum exposure of the cells to the novel editing system has three main benefits: it provides time for the homologous recombination event to occur before Cas9 is expressed, it reduces the opportunity for potential off target effects and it diminishes the likelihood of mutations that inactivate Cas9 or the sgRNA; these mutations result in “escaper” colonies that are indistinguishable from non-edited cells. Because riboswitches can be adjusted to the desired regulatory window, a riboswitch-based CRISPR system allows very low basal expression of Cas9 in the absence of inducer and an adequate level of gene expression after induction, fulfilling the benefits of an inducible Cas9-based system.

As the field of synthetic biology progresses towards more practical applications, the technologies that have been developed and optimized for canonical organisms such as E. coli are likely to be needed in other biological chassis, including Clostridium spp. This study provides a new set of tools for the efficient manipulation of industrially-relevant organisms as well as for the study of Clostridium pathogens and the development of associated therapies.

1. A genetic construct comprising a DNA polynucleotide sequence which encodes a riboswitch operably linked to a coding region, wherein the coding region encodes a target gene and the riboswitch modulates translation or transcription of the coding region.

2. The genetic construct of claim 1 wherein the target gene is a gene where background levels of expression, even at a low level, can cause harm to the cell.

3. The genetic construct of claim 1 or claim 2 wherein the riboswitch in the genetic construct may reduce the background level of target gene expression by about 5%, 10%, 20%, 15%, 20%, 25%, 30%, 35%, 40%, 45%, 50%, 55%, 60%, 65%, 70%, 75%, 80%, 85%, 90%, 95% or more.

4. The genetic construct of claim 1 or claim 2 wherein the riboswitch in the genetic construct may eliminate detectable background expression.

5. The genetic construct of any preceding claim wherein the dynamic range of expression between the riboswitch being off and the riboswitch being on, and gene expression being activated, is low.

6. The genetic construct of claim 5 wherein the dynamic range is between about 10 and about 100 fold of the off level.

7. The genetic construct of any preceding claim wherein the riboswitch is 5′ to the coding region.

8. The genetic construct of any preceding claim wherein the riboswitch comprises an aptamer domain which is capable of specifically binding to an inducer, and an expression platform which undergoes a conformational change in response to the binding of the inducer to the aptamer domain that promotes translation of the coding region.

9. The genetic construct of any preceding claim wherein the riboswitch is a naturally-occurring riboswitch or a synthetic riboswitch.

10. The genetic construct of claim 9 wherein the riboswitch specifically binds the inducer theophylline.

11. The genetic construct of claim 10 wherein the riboswitch is a positive regulatory theophylline-responsive riboswitch, and optionally has the nucleotide sequence of SEQ ID NO. 1, 2, 3, 4, 5, 6 or 7.

12. The genetic construct of claim 11 wherein the riboswitch has the nucleotide sequence of SEQ ID NO. 2.

13. The genetic construct of any preceding claim wherein the target gene is an endonuclease.

14. The genetic construct of claim 13 wherein the endonuclease can be used in genome-editing, and optionally wherein the endonuclease is a Zinc Finger Nuclease (ZFN), Transcription Activator-like Effector Nuclease (TALEN), Clustered Regularly Interspaced Short Palindromic Repeats (CRISPR) nuclease, homing meganuclease or standard restriction endonuclease (RE).

15. The genetic construct of claim 14 wherein the endonuclease is Cas9, Cas9 nickase, dCas, Cpf1, C2c1, C2c2, C2c3, a Cas9 derivative, or any endonuclease suitable for use with CRISPR gene editing, or a homolog or functional variant thereof.

16. The genetic construct of any of claims 1 to 12 wherein the target gene is a sigma factor.

17. The genetic construct of claim 16 wherein the sigma factors is TcdR (from Clostridium difficile), BotR (from Clostridium botulinum), TetR (from Clostridium tetani) or UviA (from Clostridium perfringens).

18. A vector comprising the genetic construct of any preceding claim.

19. A host cell comprising the genetic construct of any of claims 1 to 17 or a vector according to claim 18.

20. The host cell of claim 19 wherein the cell is bacterium.

21. The host cell of claim 20 wherein the bacterium is of the genus Bacillus or Clostridium.

22. A kit for regulating expression of a target gene, wherein the kit comprises the genetic construct of any of claims 1 to 17.

23. A method of controlling expression of a target gene in a cell comprising:
i) transforming a host cell with a genetic construct, the construct comprising polynucleotide to be transcribed, wherein the polynucleotide comprise is a coding region encoding a target gene operably linked to a riboswitch;
ii) exposing the transformed cell to an inducer of the riboswitch thereby effecting expression of the target gene.

24. A method of controlling expression of a target gene in a cell comprising:
i) providing a host cell which contained a genetic construct, the construct comprising polynucleotide to be transcribed, wherein the polynucleotide comprise is a coding region encoding a target gene operably linked to a riboswitch;
ii) exposing the transformed cell to an inducer of the riboswitch thereby effecting expression of the target gene.


SEQUENCE LISTING (143 sequences)

SEQ ID NO: 1 - RNA, 60 nt, Artificial Sequence
  riboswitch
ggugauaccagcaucgucuugaugcccuuggcagcacccugcuaagguaacaacaagaug

SEQ ID NO: 2 - RNA, 63 nt, Artificial Sequence
  riboswitch
ggugauaccagcaucgucuugaugcccuuggcagcacccugcuaaggagguaacaacaagaug

SEQ ID NO: 3 - RNA, 60 nt, Artificial Sequence
  riboswitch
ggugauaccagcaucgucuugaugcccuuggcagcacccugcuaaggaggcaacaacaug

SEQ ID NO: 4 - RNA, 60 nt, Artificial Sequence
  riboswitch
ggugauaccagcaucgucuugaugcccuuggcagcacccugcuaaggagguaacaacaug

SEQ ID NO: 5 - RNA, 60 nt, Artificial Sequence
  riboswitch
ggugauaccagcaucgucuugaugcccuuggcagcacccugcuaaggagguaacuuaaug

SEQ ID NO: 6 - RNA, 60 nt, Artificial Sequence
  riboswitch
ggugauaccagcaucgucuugaugcccuuggcagcacccugcuaaggagguguguuaaug

SEQ ID NO: 7 - RNA, 61 nt, Artificial Sequence
  riboswitch
ggugauaccagcaucgucuugaugcccuuggcagcacccugcuaaggaggucaacaagaug

SEQ ID NO: 8 - RNA, 35 nt, Clostridium sporogenes
aucuuuccuccacuaggucggcguccaagaggaug

SEQ ID NO: 9 - RNA, 11 nt, Clostridium sporogenes
aaaggaggugu

SEQ ID NO: 10 - protein, 9 aa, Artificial Sequence
  motif
LAGLIDADG

SEQ ID NO: 11 - DNA, 24 bp, Artificial Sequence
  recognition site
ttgaggaggtttctctgtaaataa

SEQ ID NO: 12 - DNA, 26 bp, Artificial Sequence
  recognition site
taactataacggtcctaaggtagcga

SEQ ID NO: 13 - DNA, 30 bp, Artificial Sequence
  recognition site
gaaggtttggcacctcgatgtcggctcatc

SEQ ID NO: 14 - DNA, 23 bp, Artificial Sequence
  recognition site
cgatcctaaggtagcgaaattca

SEQ ID NO: 15 - DNA, 20 bp, Artificial Sequence
  recognition site
cccggctaactctgtgccag

SEQ ID NO: 16 - DNA, 30 bp, Artificial Sequence
  recognition site
ctgggttcaaaacgtcgtgagacagtttgg

SEQ ID NO: 17 - DNA, 30 bp, Artificial Sequence
  recognition site
atgccttgccgggtaagttccggcgcgcat

SEQ ID NO: 18 - DNA, 24 bp, Artificial Sequence
  recognition site
caaaacgtcgtaagttccggcgcg

SEQ ID NO: 19 - DNA, 24 bp, Artificial Sequence
  recognition site
agtaatgagcctaacgctcagcaa

SEQ ID NO: 20 - DNA, 24 bp, Artificial Sequence
  recognition site
agtaatgagcctaacgctcaacaa

SEQ ID NO: 21 - DNA, 24 bp, Artificial Sequence
  recognition site
cacatccataaccatatcattttt

SEQ ID NO: 22 - DNA, 30 bp, Artificial Sequence; identical to SEQ ID NO: 16
  recognition site

SEQ ID NO: 23 - DNA, 30 bp, Artificial Sequence
  recognition site
gaagatgggaggagggaccggactcaactt

SEQ ID NO: 24 - DNA, 15 bp, Artificial Sequence
  recognition site
cagtactacggttac

SEQ ID NO: 25 - DNA, 25 bp, Artificial Sequence
  recognition site
gcgagcccgtaagggtgtgtacggg

SEQ ID NO: 26 - DNA, 29 bp, Artificial Sequence
  recognition site
taactatgactctcttaaggtagccaaat

SEQ ID NO: 27 - DNA, 30 bp, Artificial Sequence
  recognition site
tggcaaacagctattatgggtattatgggt

SEQ ID NO: 28 - DNA, 28 bp, Artificial Sequence
  recognition site
tgtcacattgaggtgcactagttattac

SEQ ID NO: 29 - DNA, 30 bp, Artificial Sequence
  recognition site
agttacgctagggataacagggtaatatag

SEQ ID NO: 30 - DNA, 39 bp, Artificial Sequence
  recognition site
atctatgtcgggtgcggagaaagaggtaatgaaatggca

SEQ ID NO: 31 - DNA, 29 bp, Artificial Sequence
  recognition site
ttttgattctttggtcaccctgaagtata

SEQ ID NO: 32 - DNA, 29 bp, Artificial Sequence
  recognition site
attggaggttttggtaactatttattacc

SEQ ID NO: 33 - DNA, 29 bp, Artificial Sequence
  recognition site
tcttttctcttgattagccctaatctacg

SEQ ID NO: 34 - DNA, 24 bp, Artificial Sequence
  recognition aite
aataattttcttcttagtaatgcc

SEQ ID NO: 35 - DNA, 24 bp, Artificial Sequence
  recognition site
gttatttaatgttttagtagttgg

SEQ ID NO: 36 - DNA, 28 bp, Artificial Sequence
  recognition site
tgtcacattgaggtgcactagttattac

SEQ ID NO: 37 - DNA, 19 bp, Artificial Sequence
  recognition site
gtcgggctcataacccgaa

SEQ ID NO: 38 - DNA, 24 bp, Artificial Sequence
  recognition site
agtggtatcaacgctcagtagatg

SEQ ID NO: 39 - DNA, 30 bp, Artificial Sequence
  recognition site
gcttatgagtatgaagtgaacacgttattc

SEQ ID NO: 40 - DNA, 30 bp, Artificial Sequence
  recognition site
tatgtatcttttgcgtgtacctttaacttc

SEQ ID NO: 41 - DNA, 22 bp, Artificial Sequence
  recognition site
  misc_feature (6)..(6): n is a, c, g, or t
  misc_feature (12)..(12): n is a, c, g, or t
taygcngayacngacggyttyt

SEQ ID NO: 42 - DNA, 30 bp, Artificial Sequence
  recognition site
aaattgcttgcaaacagctattacggctat

SEQ ID NO: 43 - DNA, 19 bp, Artificial Sequence
  recognition site
cttcagtatgccccgaaac

SEQ ID NO: 44 - DNA, 24 bp, Artificial Sequence
  recognition site
cctgactctcttaaggtagccaaa

SEQ ID NO: 45 - DNA, 37 bp, Artificial Sequence
  primer
atatgcggccgcgtgtagtagcctgtgaaataagtaa

SEQ ID NO: 46 - DNA, 40 bp, Artificial Sequence
  primer
atatggtgtcaccattaacacacctccttaaaaattacac

SEQ ID NO: 47 - DNA, 28 bp, Artificial Sequence
  primer
tataggtctcaatggtatttgaaaaaat

SEQ ID NO: 48 - DNA, 31 bp, Artificial Sequence
  primer
tatactcgagttaactatttatcaattcctg

SEQ ID NO: 49 - DNA, 36 bp, Artificial Sequence
  primer
cagcggccgcatggtatttgaaaaaattgataaaaa

SEQ ID NO: 50 - DNA, 40 bp, Artificial Sequence
  primer
ggcggtaccgatttatattttaccatgtttttattaattt

SEQ ID NO: 51 - DNA, 61 bp, Artificial Sequence
  primer
cccggtaccaatacgactcactataggttccggtgataccagcatcgtcttgatgcccttg

SEQ ID NO: 52 - DNA, 61 bp, Artificial Sequence
  primer
ccggtctcaccatgttgttgcctccttagcagggtgctgccaagggcatcaagacgatgct

SEQ ID NO: 53 - DNA, 61 bp, Artificial Sequence; identical to SEQ ID NO: 52
  primer

SEQ ID NO: 54 - DNA, 63 bp, Artificial Sequence
  primer
ccggtctcaccatcttgttgttacctccttagcagggtgctgccaagggcatcaagacgatgc

SEQ ID NO: 55 - DNA, 59 bp, Artificial Sequence
  primer
ccggtctcaccatgttgttacctccttagcagggtgctgccaagggcatcaagacgatg

SEQ ID NO: 56 - DNA, 60 bp, Artificial Sequence
  primer
ccggtctcaccattaagttacctccttagcagggtgctgccaagggcatcaagacgatgc

SEQ ID NO: 57 - DNA, 60 bp, Artificial Sequence
  primer
ccggtctcaccattaacacacctccttagcagggtgctgccaagggcatcaagacgatgc

SEQ ID NO: 58 - DNA, 61 bp, Artificial Sequence
  primer
ccggtctcaccatcttgttgacctccttagcagggtgctgccaagggcatcaagacgatgc

SEQ ID NO: 59 - DNA, 63 bp, Artificial Sequence
  primer
ccggtctcaccattggtcgtacactcccttagcagggtgctgccaagggcatcaagacgatgc

SEQ ID NO: 60 - DNA, 42 bp, Artificial Sequence
  primer
cccggtctctattgaaaaattacacaactttatacgatttat

SEQ ID NO: 61 - DNA, 54 bp, Artificial Sequence
  primer
ggtctcacaatacgactcactataggtgataccagcatcgtcttgatgcccttg

SEQ ID NO: 62 - DNA, 59 bp, Artificial Sequence
  primer
aaacttaacttcatgttaaaaacttgttaaaatataaatcgtataaagttgtgtaattt

SEQ ID NO: 63 - DNA, 60 bp, Artificial Sequence
  primer
atttatattttaacaagtttttaacatgaagttaagttttttttatataactgtataacc

SEQ ID NO: 64 - DNA, 66 bp, Artificial Sequence
  primer
atatatatgcggccgctgtatccatatgaccatgattacgaattctcagtcacctcctagctgact

SEQ ID NO: 65 - DNA, 40 bp, Artificial Sequence
  primer
gctatggtctccatggataagaaatactcaataggcttag

SEQ ID NO: 66 - DNA, 67 bp, Artificial Sequence
  primer
ggcctctagagtgtagtagcctgtgaaataagtaaggaaaaaaaagaagtaagtgttatatatgatg

SEQ ID NO: 67 - DNA, 59 bp, Artificial Sequence
  primer
ggccggcgcgccgtgtagtagcctgtgaaataagtaaggaaaaaaaagaagtaagtgtt

SEQ ID NO: 68 - DNA, 32 bp, Artificial Sequence
  primer
atatattctagatttatatttagtcccttgcc

SEQ ID NO: 69 - DNA, 48 bp, Artificial Sequence
  primer
ggccggcgcgccattgacgtctatgtcgacgaaaactcctccttaaga

SEQ ID NO: 70 - DNA, 64 bp, Artificial Sequence
  primer
gtatcatctagagtaaaacgacggccagtttgacagctagctcagtcctaggtataatactagt

SEQ ID NO: 71 - DNA, 66 bp, Artificial Sequence
  primer
gtatcaggcgcgccattgacgtctatactagtattatacctaggactgagctagctgtcaaactgg

SEQ ID NO: 72 - DNA, 93 bp, Artificial Sequence
  primer
ggccccgggacgtcataaaaataagaagcctgcaaatgcaggcttcttatttttataaaaaaagcaccgactcggtgccactttttcaagttg

SEQ ID NO: 73 - DNA, 44 bp, Artificial Sequence
  primer
ggccgacgtctcattagatgcatattcaatgcaggatagtatag

SEQ ID NO: 74 - DNA, 60 bp, Artificial Sequence
  primer
attttcaatacagaggttgatcttatttattagttattattaccaaattttatagttata

SEQ ID NO: 75 - DNA, 55 bp, Artificial Sequence
  primer
ataaataagatcaacctctgtattgaaaataatacagagtttttttataataaaa

SEQ ID NO: 76 - DNA, 56 bp, Artificial Sequence
  primer
aaggcggcgcgccattatgaactacaaactttctcatttaatagatgaatttgacc

SEQ ID NO: 77 - DNA, 113 bp, Artificial Sequence
  primer
ccgggtcgacggtaatataacattgccacagttttagagctagaaatagcaagttaaaataaggctagtccgttatcaacttgaaaaagtggcaccgagtcggtgcttttttt

SEQ ID NO: 78 - DNA, 113 bp, Artificial Sequence
  primer
ggccactagtggtaatataacattgccacagttttagagctagaaatagcaagttaaaataaggctagtccgttatcaacttgaaaaagtggcaccgagtcggtgcttttttt

SEQ ID NO: 79 - DNA, 31 bp, Artificial Sequence
  primer
tttccgttctaggagtggatgcgggaaagga

SEQ ID NO: 80 - DNA, 31 bp, Artificial Sequence
  primer
tgaccattcagaggatgatgaaggtttagcc

SEQ ID NO: 81 - DNA, 68 bp, Artificial Sequence
  primer
ggccggcgcgccgctggagtggcggaactggcagacgcacaggacttaaaatcctgcgggcctaacag

SEQ ID NO: 82 - DNA, 76 bp, Artificial Sequence
  primer
tatattctctttctatattttaatataattgcataataattaatcacccccattaagttacttctcaattatatca

SEQ ID NO: 83 - DNA, 79 bp, Artificial Sequence
  primer
acttaatgggggtgattaattattatgcaattatattaaaatatagaaagagaatataaagttaagtagtaaatactta

SEQ ID NO: 84 - DNA, 83 bp, Artificial Sequence
  primer
ggccgacgtctcatcatcaatatttttcacaataattttctatatcttctcttaatacatttattaaaggtcttatatataca

SEQ ID NO: 85 - DNA, 113 bp, Artificial Sequence
  primer
ggccgtcgacttaattataggatatgtaaagttttagagctagaaatagcaagttaaaataaggctagtccgttatcaacttgaaaaagtggcaccgagtcggtgcttttttt

SEQ ID NO: 86 - DNA, 31 bp, Artificial Sequence
  primer
atataattatttatgtcgtcagcaatgagac

SEQ ID NO: 87 - DNA, 29 bp, Artificial Sequence
  primer
ctctcttatctattattaccctttgtgtt

SEQ ID NO: 88 - DNA, 42 bp, Artificial Sequence
  primer
ggccgacgtcatgcaatatggcgcagaacttttaccatacca

SEQ ID NO: 89 - DNA, 58 bp, Artificial Sequence
  primer
ggccgctcttctaagtcatcatcatatcatagtaatagatattaaattcattaaattt

SEQ ID NO: 90 - DNA, 96 bp, Artificial Sequence
  primer
ggccgctcttccctttaaggaggtgtgttacatatgaacaaaaatataaaatattctcaaaactttttaacgagtgaaaaagtactcaaccaaata

SEQ ID NO: 91 - DNA, 79 bp, Artificial Sequence
  primer
gtaccgagctcgaattcgtaatttattattatttcctcccgttaaataatagataactattaaaaatagacaatacttg

SEQ ID NO: 92 - DNA, 86 bp, Artificial Sequence
  primer
tctattatttaacgggaggaaataataataaattacgaattcgagctcggtacccggggatcctctagagtcgacgtcacgcgtcc

SEQ ID NO: 93 - DNA, 109 bp, Artificial Sequence
  primer
aacacacctccttaaatcactgcccgctttccagtcgggaaaccctagcgccattcgccattcaggctgcgcaactgttgggaagggcgatcggtgcgggcctcttcgc

SEQ ID NO: 94 - DNA, 94 bp, Artificial Sequence
  primer
ggtttcccgactggaaagcgggcagtgatttaaggaggtgtgttacatatgttacgtcctgtagaaaccccaacccgtgaaatcaaaaaactcg

SEQ ID NO: 95 - DNA, 51 bp, Artificial Sequence
  primer
gctcttctcattgtttgcctccctgctgcggtttttcaccgaagttcatgc

SEQ ID NO: 96 - DNA, 50 bp, Artificial Sequence
  primer
ggccgctcttccatgagggaagtactagtgcttcagctatcggtgtggct

SEQ ID NO: 97 - DNA, 46 bp, Artificial Sequence
  primer
ggccggcgcgcctttcatttattatataattatttactatatcttc

SEQ ID NO: 98 - DNA, 76 bp, Artificial Sequence
  primer
ggccggcgcgccgccgaagtggcggaactggcagacgcacaggacttaaaatcctgcggtgcttaaaccaccgtac

SEQ ID NO: 99 - DNA, 74 bp, Artificial Sequence
  primer
attcaattaataaacaacattttaatacaattgcatacaaacatctcccaacctataaactatttttattataa

SEQ ID NO: 100 - DNA, 72 bp, Artificial Sequence
  primer
gtttataggttgggagatgtttgtatgcaattgtattaaaatgttgtttattaattgaatgagaatttataa

SEQ ID NO: 101 - DNA, 77 bp, Artificial Sequence
  primer
ggccgacgtctcatcatcatggattaatgtgttccttcttacaatagctttctatttcttctcttcctatttttatt

SEQ ID NO: 102 - DNA, 113 bp, Artificial Sequence
  primer
ggccgtcgacggaaactcctaaatatcacggttttagagctagaaatagcaagttaaaataaggctagtccgttatcaacttgaaaaagtggcaccgagtcggtgcttttttt

SEQ ID NO: 103 - DNA, 30 bp, Artificial Sequence
  primer
atgatatataatagtttctgttgcagtaag

SEQ ID NO: 104 - DNA, 29 bp, Artificial Sequence
  primer
gttatctacatttactgtatcagataatc

SEQ ID NO: 105 - DNA, 76 bp, Artificial Sequence
  primer
ggccggcgcgccgttacgggaatagaagaatcaaatttgggctttaagggtgttttagaaggaatacaaggtgatg

SEQ ID NO: 106 - DNA, 75 bp, Artificial Sequence
  primer
atacacataatcttgtaaaccttattttgtttgcataaaaaaacctcctcttttgtttatattaagaattgtaac

SEQ ID NO: 107 - DNA, 77 bp, Artificial Sequence
  primer
ataaacaaaagaggaggtttttttatgcaaacaaaataaggtttacaagattatgtgtataagatgtggataacttg

SEQ ID NO: 108 - DNA, 77 bp, Artificial Sequence
  primer
ggccgacgtctcacctcatcaatcaagtttttctttaatgtttaaggcaaaagacaaatgtgttggcgattttgtta

SEQ ID NO: 109 - DNA, 113 bp, Artificial Sequence
  primer
ggccgtcgacggaattttagatttaaaacggttttagagctagaaatagcaagttaaaataaggctagtccgttatcaacttgaaaaagtggcaccgagtcggtgcttttttt

SEQ ID NO: 110 - DNA, 29 bp, Artificial Sequence
  primer
ttctgataagtgtaatgatgaaaagtgtg

SEQ ID NO: 111 - DNA, 29 bp, Artificial Sequence
  primer
ctaactctaaatctgaacaaatccccata

SEQ ID NO: 112 - DNA, 50 bp, Artificial Sequence
  primer
gttcatggcgcgcccttctaaatagttattgtctttagaaactatattag

SEQ ID NO: 113 - DNA, 51 bp, Artificial Sequence
  primer
gctatggtctccttatactaaaatatagaaagagaatataaagttaggtag

SEQ ID NO: 114 - DNA, 46 bp, Artificial Sequence
  primer
gctactggtctcaataattgcataataattaatcacccccattaag

SEQ ID NO: 115 - DNA, 37 bp, Artificial Sequence
  primer
gtagccgacgtccagcgatgagacataaacataaaac

SEQ ID NO: 116 - DNA, 107 bp, Artificial Sequence
  primer
ttttcgtcgacttttataaagcgaggaactggttttagagctagaaatagcaagttaaaataaggctagtccgttatcaacttgaaaaagtggcaccgagtcggtgc

SEQ ID NO: 117 - DNA, 29 bp, Artificial Sequence
  primer
gaaataaagtgttgacatagtaaagtttg

SEQ ID NO: 118 - DNA, 29 bp, Artificial Sequence
  primer
aaagctctcttatctattattaccctttg

SEQ ID NO: 119 - DNA, 21 bp, Artificial Sequence
  primer
ctactttgcaagtgtaccttg

SEQ ID NO: 120 - DNA, 20 bp, Artificial Sequence
  primer
atccccaattcaccatcttg

SEQ ID NO: 121 - DNA, 20 bp, Artificial Sequence
  primer
agcggtgaaatgcgtagaga

SEQ ID NO: 122 - DNA, 20 bp, Artificial Sequence
  primer
ggcacagggggagttgatac

SEQ ID NO: 123 - DNA, 22 bp, Artificial Sequence
  primer
gaaagggtattcaagccacatc

SEQ ID NO: 124 - DNA, 23 bp, Artificial Sequence
  primer
ctatcggtgtggctatgggggct

SEQ ID NO: 125 - DNA, 23 bp, Artificial Sequence
  primer
aaatgtgaaatccgtcacatact

SEQ ID NO: 126 - DNA, 27 bp, Artificial Sequence
  primer
caaaggaagtataattttgttatcttc

SEQ ID NO: 127 - DNA, 29 bp, Artificial Sequence
  primer
ctgctagctttctgcaaattcagattaaa

SEQ ID NO: 128 - DNA, 20 bp, Artificial Sequence
  spacer
ggaaactcctaaatatcacg

SEQ ID NO: 129 - DNA, 20 bp, Artificial Sequence
  spacer
ggaattttagatttaaaacg

SEQ ID NO: 130 - DNA, 20 bp, Artificial Sequence
  spacer
ttttataaagcgaggaactg

SEQ ID NO: 131 - DNA, 20 bp, Artificial Sequence
  spacer
ttaattataggatatgtaaa

SEQ ID NO: 132 - DNA, 20 bp, Artificial Sequence
  spaver
ttaattataggatatgtaaa

SEQ ID NO: 133 - DNA, 20 bp, Artificial Sequence
  spacer
ggtaatataacattgccaca

SEQ ID NO: 134 - DNA, 20 bp, Artificial Sequence
  primer
acagttttttcaattttcgg

SEQ ID NO: 135 - DNA, 20 bp, Artificial Sequence
  spacer
ttacaactactatccaaaga

SEQ ID NO: 136 - DNA, 20 bp, Artificial Sequence
  spacer
aagtactccgattaaaacag

SEQ ID NO: 137 - DNA, 20 bp, Artificial Sequence
  spacer
ggtttagaagaaatagcggg

SEQ ID NO: 138 - DNA, 20 bp, Artificial Sequence
  spacer
gacaaaacaagtcaaataga

SEQ ID NO: 139 - DNA, 20 bp, Artificial Sequence
  spacer
ggactcttaaccccaactaa

SEQ ID NO: 140 - DNA, 20 bp, Artificial Sequence
  spacer
ccaaatatagtccaacctaa

SEQ ID NO: 141 - skipped

SEQ ID NO: 142 - DNA, 20 bp, Artificial Sequence
  spacer
gttgttttaattgatcacaa

SEQ ID NO: 143 - DNA, 20 bp, Artificial Sequence
  spacer
acttgtagcggcagaagcac